(19) United States
(12) Patent Application Publication
(10) Pub. No.: US 2018/0111977 A1
(43) Pub. Date: Apr. 26, 2018

(54) RECOMBINANT SOLUBLE HUMAN TISSUE FACTOR, METHOD OF ITS PRODUCTION AND USES THEREOF

(71) Applicant: Dana Genetic A/S, Gentofte (DK)

(72) Inventor: Kurt Bækgaard Osther, Scottsdale, AZ (US)

(73) Assignee: Dana Genetic A/S, Gentofte (DK)

(21) Appl. No.: 15/561,167

(22) PCT Filed: Mar. 23, 2016
(86) PCT No.: PCT/DK2016/050090

§ 371 Date: Sep. 25, 2017

Related U.S. Application Data
(60) Provisional application No. 62/177,776, filed on Mar. 23, 2015.
(60) Provisional application No. 62/285,359, filed on Oct. 26, 2015.

(51) Int. Cl. C07K 14/745; A61P 7/04; A61K 9/00; C12N 5/073

(52) U.S. Cl. CPC C07K 14/745; A61P 7/04; A61K 38/00; C12N 5/0603; A61K 9/0014

(57) ABSTRACT

The present invention relates to a novel recombinant soluble human Tissue Factor(RshTF), which is an extrinsic hemostasis factor, produced in human HEK-cells. The present invention also relates to a hitherto unknown method of producing the RshTF as well as specific uses of RshTF and protein compositions comprising RshTF.

### TECHNICAL FIELD OF THE INVENTION

The present invention relates to a novel recombinant soluble human Tissue Factor (RshTF), which is an extrinsic hemostasis factor, produced in Human Embryonic Kidney cells (HEK-cells). The present invention also relates to novel method of producing the RshTF as well as specific uses of RshTF and protein compositions comprising RshTF.

### BACKGROUND OF THE INVENTION

Blood clotting, or coagulation, is a rapid response to tissue damage. Endogenous Tissue Factor from the damaged cells from the blood vessel walls is often not sufficient to initiate a rapid cascade system. Factor III is a profoundly important exogenous component to obtain efficient and fast hemostasis when applied to a bleeding or excessive bleeding lesion

As is known in the art, in order to stop the bleeding of for example severely bleeding war victims the traditional treatment has been with relatively labile blood clotting enzymes such as thrombin. Such treatments have typically been carried out on trauma sites in a military combat zone. However, it is well-known that around 40% of severely bleeding.

As is known in the art, in order to stop the bleeding of for example severely bleeding war victims the traditional treatment has been with relatively labile blood clotting enzymes such as thrombin, which has not proven efficient to be useable for hemostatic treatment at any trauma site, e.g. due to the instability of thrombin. Such treatments typically have to be brought to any trauma site, where no hemostatic products such as intrinsic or extrinsic clotting protein have been available to retain clotting activity so that they could be carried out on trauma sites in a military combat zone, or any trauma site at all.

However, it is well-known that around 40% of severely bleeding has a fatal outcome. A blood-clotting agent that can cause soluble fibrinogen to be converted to insoluble fibrin has not successfully been used on severely bleeding conditions due to stability problems of such an agent, which in turn affects negatively the activity of said agent.

More specifically, the present invention concerns the extracellular part of the RshTF and a method of producing said RshTF in human HEK-cells, which differs significant from the previously prepared recombinant Tissue Factors made in e.g. E. coli containing no glycosylation and being relatively unstable. Other examples or production methods known in the art are production of Tissue Factor in Chinese Hamster Ovary (CHO) Cells, where the proteins undergo post-translational modification (PTM), which can alter the physical and chemical properties of the resulting Tissue Factor, e.g., in relation to molecular weight (MW), folding, stability, and biological activity.

Another drawback in the art is the fact that other recombinant Tissue Factors, such as Tissue Factors produced in e.g. E. coli, is a recombinant produced single, non-glycosylated polypeptide chain not having sufficient stability for a reasonable time period, as for instance more than 1 month, because it is known that such Tissue Factors have to be stored at approx. 4° C. and would under those conditions have to be used within 2-4 weeks. Storage for longer periods would require freezing at −20° C. or below. Moreover, recombinant Tissue Factors produced in E. coli cannot tolerate freeze thaw cycles.

It is well-known that antibody response elicited to and against material (antigen) produced in xenogeneic cells such as for instance CHO-cells (Chinese hamster ovary cells) or BHK-cells (Baby hamster kidney cells) was higher than that to human expressed material. It is also known in the art that CHO-expressed material is more immunogenic in mice than corresponding material expressed in human cells.

Hence, human transfected host cells will produce a human glycosylation, and will not induce antibodies against human glycosylation. Furthermore, the protein yield in a human expression system, such as the HEK-systems according to the present invention, is often fold or more than the yields when produced in xenogeneic cells such as CHO-cell. These are some of the factors causing the problems in relation to both yield and antigenicity in number commercial thrombin products such as e.g. Recothromb.

The stable RshTF-comprising products of the present invention, which can tolerate ambient temperatures without losing activity, because this product is a glycoprotein without any protease or enzyme structure, therefore solves the above problems in the art. Therefore, products comprising the RshTF of the present invention have a broad area of potential uses ranging from applications at trauma sites to controlled treatment sites, hospitals, clinics, etc. Products comprising the RshTF of the present invention can also be used anywhere where clotting products such as thrombin can be used.

### SUMMARY OF THE INVENTION

An object of the present invention relates to the provision of a stable blood clotting agent which tolerates ambient temperatures without losing activity, is easy to produce, can be produced in high yields and having low antigenicity when used to treat and/or prevent bleedings in humans.

In particular, it is an object of the present invention to provide a RshTF comprising the SEQ ID NO: 2, wherein the RshTF is produced in a human cell, that solves the above mentioned problems of the prior art in relation to traditionally used blood clotting agents.

Thus, one aspect of the invention relates to a RshTF comprising the SEQ ID NO: 2, wherein the RshTF is produced in a human cell, preferably a HEK-cell.

Another aspect of the present invention relates to a protein composition comprising RshTF in combination with one or more intrinsic factor, such as e.g. Factor VII/VIIa, participating in blood clotting or hemostasis.

Yet another aspect of the present invention is to provide a kit-of parts comprising an essentially air- and humidity-tight receptacle comprising a scaffold impregnated and/or coated with RshTF or a protein composition comprising RshTF, and optionally a humidity absorbing material.

Still another aspect of the present invention is to provide a method of producing RshTF, said method comprising culturing in vitro a human host cell transfected with at least one exogenous nucleic acid sequence encoding said RshTF, wherein the human host cell is a human HEK cell.

### BRIEF DESCRIPTION OF THE FIGURES

FIG. 1 shows a damaged vessel, where a scaffold coated with RshTF. The release of Tissue Factor (TF) in contact with Factor VII, then Factor VII will be converted to TF/Factor VIIa, which via the co-factor, Factor VIIIa activates Factor IX to Factor IXa, which again activates Factor X to Xa,—or directly from the RshTF:Factor VII/VIIa to Factor Xa. Factor Xa activates (via co-factor Va) Factor II (prothrombin) to Factor IIa, and Factor IIa (thrombin) converts soluble fibrinogen to fibrin (the clotting gets somewhat more firm, with the presence of another endogen factor called Factor XIII.

FIG. 2 shows a schematic diagram of human Tissue Factor (263 amino acids) with extracellular, transmembrane, and cytoplasmic domains, and soluble Tissue Factor (219 amino acids) with extracellular domain only. The extracellular domain of soluble TF has 3 glycosylation sites and two intramolecular disulfide bonds as in the diagram.

FIG. 3 shows the binding of RshTF to Factor VII/VIIa. RshTF:Factor VII/VIIa actvates Factor Xa (and Factor IXa) to activate Factor II (prothrombin) to Factor II(a) thrombin.

FIG. 4 shows pST2 vector for cloning and pST2-HF3 after cloning. pST2-HF3 was linearized by BglII and PciI restriction enzymes. pST2-HF3 nucleotide full sequence is in SEQ ID NO: 5.

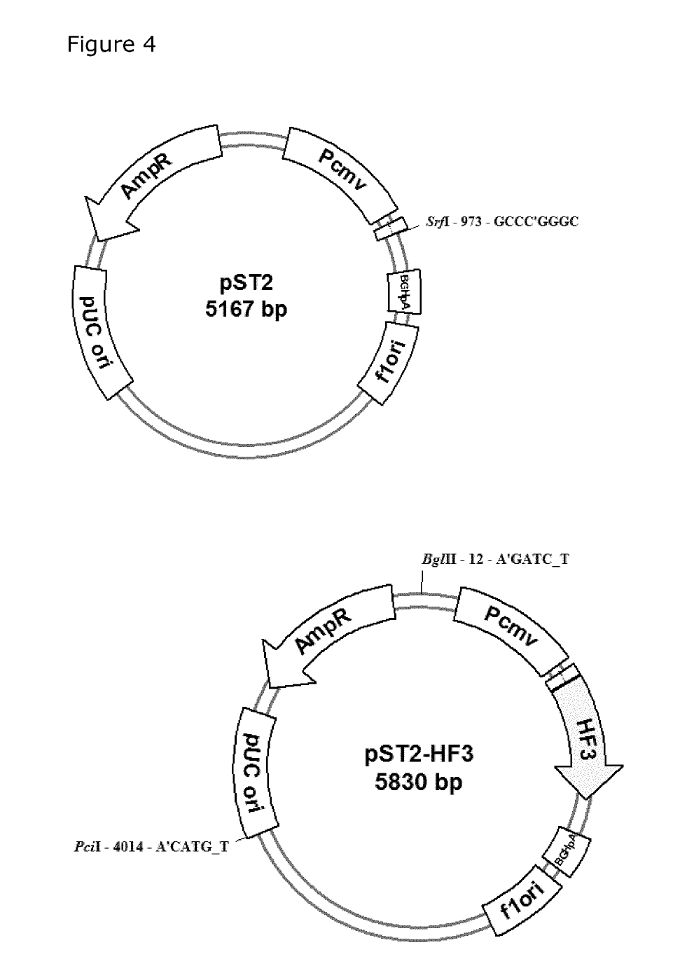

FIG. 5 shows typical HEK293T cell morphologies grown in monolayer culture on a 100 mm Petri dish, see above photo (A); and HEK293T cells in serum-free culture medium in suspension culture in a shaking flask at 37° C., the HEK293T cells appear round in shape, see below photo (B).

FIG. 6 shows identification of RshTF expression from serum-free and suspension adapted cell pools on SDS-PAGE (6A) and by Western blot as shown as reduced and non reduced (6B).

FIG. 7 shows HEK293T culture (1 Litre) of RshTF cell lines was expanded in serum-free, chemically defined medium at 37° C. with improved expression level. During (nine) 9 days cell culturing, the protein expression level was monitored on SDS-PAGE (FIG. 7(A)), and viable cell density and viability were recorded (FIG. 7(B)) on day 2, 3, 5, 7, and day 9.

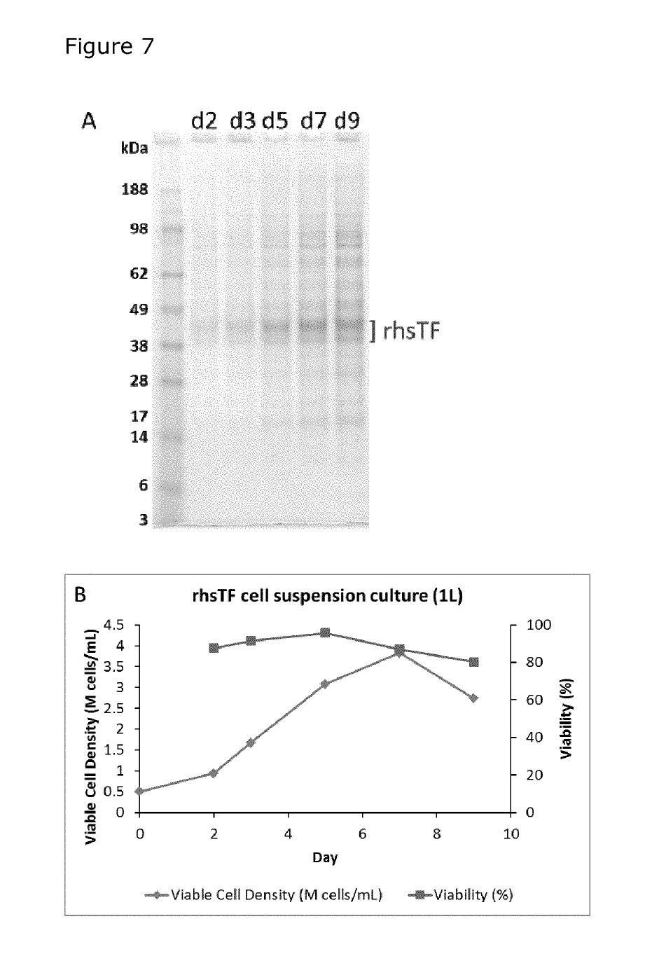

FIG. 8 shows blood clot formation in human whole blood by mixing with RshTF (in FIG. 8(B) referred to as [RhsTF]) (2.5 μg/mL) (right) versus no clot with PBS (left) in 7 minutes; FIG. 8(B). Clot formation time was shortened by mixing with RshTF in a dose-dependent manner, where the shortest clotting time was found to be approximately 7 minutes (0 is testing using PBS as the control).

The present invention will now be described in more detail in the following.

### DETAILED DESCRIPTION OF THE INVENTION

Definitions

Prior to discussing the present invention in further details, the following terms, abbreviations and conventions will first be defined:

Injury Severity Score (ISS)

The RshTF will be tested for instance by the US Army on pigs, after a certain pig—lesion model, which imitates the Injury scale used among others by The US Army and by the US Armed Forces. The scale is as follows:

The Injury Severity Score (ISS) is an anatomical scoring system that provides an overall score for patients with multiple injuries. Each injury is assigned an Abbreviated Injury Scale (AIS) score and is allocated to one of six body regions (Head, Face, Chest, Abdomen, Extremities (including Pelvis), External). Only the highest AIS score in each body region is used. The 3 most severely injured body regions have their score squared and added together to produce the ISS score.

An example of the ISS calculation is shown below:

TABLE 1

| Region | Injury Description | AIS | Square Top Three |
|---|---|---|---|
| Head & Neck | Cerebral Contusion | 3 | 9 |
| Face | No Injury | 0 | |
| Chest | Flail Chest | 4 | 16 |
| Abdomen | Minor Contusion of Liver | 2 | 25 |
| | Complex Rupture Spleen | 5 | |
| Extremity | Fractured femur | 3 | |
| External | No Injury | 0 | |

| | |
|---|---|
| Injury Severity Score (ISS): | 50 |

The ISS score takes values from 0 to 75. If an injury is assigned an AIS of 6 (unsurvivable injury), the ISS score is automatically assigned to 75. The ISS score is virtually the only anatomical scoring system in use and correlates linearly with mortality, morbidity, hospital stay and other measures of severity.

Thus, according to the internationally recognized ISS scoring system a bleeding having an ISS value above 15 (ISS>15) is categorized a “severe bleeding”.

It should be noted that embodiments and features described in the context of one of the aspects of the present invention also apply to the other aspects of the invention.

All patent and non-patent references cited in the present application, are hereby incorporated by reference in their entirety.

The Novel Protein—RshTF

The present invention relates to a novel recombinant soluble human Tissue Factor, RshTF, produced in a human cell.

In accordance to an embodiment of the present invention, RshTF, which is a human glycoprotein, is produced and glycosylated by the machinery of the human HEK-cell, preferably HEK293t host cell, without any protease or enzyme effect.

RshTF's Role in the Blood Clotting Cascade

Damage to blood vessel walls exposes Tissue Factor-containing cells from underlying cell layers to the bloodstream, but in the event of excessive bleeding, this brief exposure as well as in too small amount to connect in sufficient amount to Factor VII/VIIa to initiate a satisfactory hemostasis, and therefore, it is often not sufficient to cause the excessive bleeding to stop.

Therefore, the RshTF of the present invention is then able to bind, in the presence of calcium, to Factor VII (FVII), which circulates at low levels in the bloodstream; the calcium forms a bridge between the extrinsic RshTF of the present invention and Factor VII.

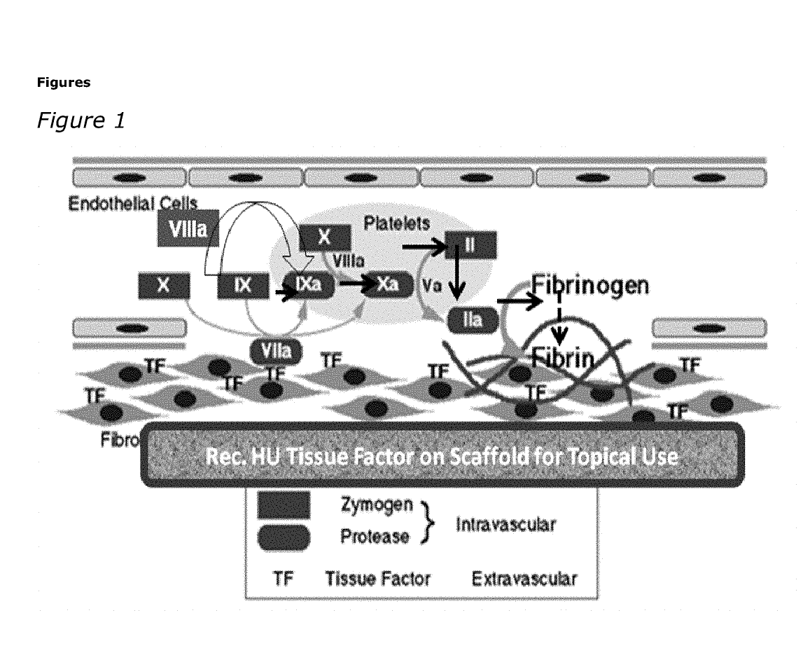

This sets off an extracellular cascade involving sequential serine protease activations: the extracellular domain of the RshTF of the present invention and FVII is activated by auto-cleavage to RshTF/FVIIa, which along with FVIIIa (cofactor) converts FIX to FIXa, which again converts FX to FXa (although TF/FVIIa can also directly convert FX to FXa), which along with FVa (cofactor) converts FII (prothrombin) to FIIa (thrombin), which converts fibrinogen to fibrin, leading to fibrin deposition and the activation of platelets to form blood clots (the activation of FXIII to FXIIIa stabilizes the fibrin clot by cross-linking it) (see FIG. 1).

So, the RshTF of the present invention interacts with Factor VII/VIIa and the activation of zymogen (intrinsic factors) which converts to proteases and via the chain of protease, Factor II (prothrormbin) is converted to Factor Ha (thrombin), which convert Factor I (fibrinogen) to fibrin.

Thus, the RshTF of the present invention has proven to bind to Factor VII/VIIa to form RshTF:VII/VIIa complex that, again, has proven to be capable of activating FXa resulting in activating of Factor II (prothrombin) to Factor IIa, thrombin.

RshTF for Use in Treatment and/or Prevention of Bleedings (Hemostasis)

In addition, the present invention relates to RshTF and RshTF comprising protein composition for use in treatment and/or prevention of bleedings (hemostasis).

RshTF according to the present invention is capable of creating hemostasis, making it possible to be used for a wide range of bleedings. For instance first aid personnel may use the RshTF or RshTF comprising protein composition of the present invention impregnated on a scaffold in a Medipack and could be used by military personnel who cannot bring autolytic products such as thrombin with them because thrombin itself decays within hours to few days without being deep frozen, and is therefore unpractical for use of first aid personnel.

The RshTF or a RshTF comprising protein composition of the present invention can, by applying suitable carriers or support materials, such as haemostatic scaffolds, activate the intrinsic system during serious trauma cases with serious excessive bleedings, when applied locally to the bleeding area, on a carrier or scaffold that can be brought into close contact with the bleeding area.

In an embodiment it may be needed to co-administer Factor VIIa concurrently with the topical application on the bleeding site of the RshTF or a RshTF comprising protein composition of the present invention.

In another embodiment it might be desirable to topically administer the RshTF or a RshTF comprising protein composition of the present invention together with soluble fibrinogen as a co-administration.

In still another embodiment it might be desirable to topically administer the RshTF or a RshTF comprising protein composition of the present invention concomitantly with one or more systemic administration(s) of Factor VII and/or Factor VIIa.

In still another embodiment it might be desirable to topically administer the RshTF or a RshTF comprising protein composition of the present invention concomitantly with adding collagen to the bleeding site.

The RshTF or a RshTF comprising protein composition of the present invention can also be used during surgery in vascular areas of the body, where the RshTF or a RshTF comprising protein composition, with or without carrier or on scaffold, alternatively mixed with certain type of powder or PEG composites, can be brought to the bleeding zone, and induce hemostasis. The thromboelastrography experiments carried out in connection to the present invention indicates that the RshTF of the present invention can be used to stop bleedings during surgery, for instance during vascular surgery, cardiovascular surgery or even in treatment and/or prevention of haemophilia.

Method of Producing RshTF

RshTF of the present invention constitutes as an important part of the Tissue Factor, the extracellular domain of the Tissue Factor, which is not an enzyme but a relatively stable glycoprotein with three glycosylation areas, which means that it is of great importance in what cell system the coding region for this part of the Tissue Factor together with its expression vector is used.

Because the transfected host cell decides what glycosylation the translated glycoprotein will show, and especially in cases where there is more than one glycosylation, Tissue Factor is extremely dependent on which host cell system is used. When the resulting protein, such as RshTF, is for use in humans, the most optimal host cells for producing said protein would of course be human cells, the machinery of which will result in human glycosylation, important for its biological function in humans as well as the important to evade any immune reaction against the glycosylated part of the recombinant protein, such as RshTF.

Thus, the RshTF of the present invention is produced by transfection into human cell lines, such as HEK—cells, preferably HEK293t cell lines, and expressed in considerable amount easy to purify.

Due to the fact that the RshTF of the present invention is a glycoprotein and not an enzyme or a protease it is already known to be significantly more stable than any of the other intrinsic factors such as for instance thrombin which is an unstable serine protease that converts soluble fibrinogen to insoluble fibrin.

In light of this, the inventors constructed an expression vector and linearized it to transfect HEK293T cells. After the transfection, stable cell lines which were growing in the presence of zeocin (a broad-spectrum antibiotic that is effective against most bacteria) were selected. The stable cell lines were adapted to grow in serum-free and suspension culture, and produced 30-40 mg/L RshTF of the present invention.

Because of the stability of the RshTF of the present invention when dried either as lyophilized or in an air dried, meaning with a minimum of solvent for instance a buffer and/or water at a low ppm, meaning minor humidity, or as a solvent, the RshTF can be buffer processed to a phosphate buffered saline (PBS) at a pH range for instance between pH 7.2-7.5 using Ultra 15 (MWCO 10 kDa, Millipore) according to the manufacturer's manual and sterilized through a 0.2 um filter. Its concentration can be measured by Agilent 8543 UV/vis spectrophotometer.

The RshTF produced by the method of the present invention may be less heterogenic, but on the contrary, more homogenous, enabling a better consistency in the final protein product, more reproducible from lot to lot, functionally more active and less immunogenic due to the lesser difference in the sugar molecules, which otherwise may be obtained in cells such as insect cells or CHO-cells. At the same time, the final product will also be virus free and free of pathologic prions.

The host cells, i.e. human HEK-cells, used in the method according to the present invention allows for a stable and high yield expression of RshTF. Said cells may be grown in suspension under serum-free conditions to high densities in different types of bioreactors.

Mammalian cells, such as human HEK-cells, are generally cultured in commercially available serum-containing or serum-free media. Selection of a medium appropriate for the particular cell line used is within the level of ordinary skill in the art.

Serum-free culture medium for HEK-cell cultures or cells lines and their suitable growth and expression media may be used to further improve protein production yields.

In an embodiment of the present invention the human host cell may be cultured to a cell density of above 10³ cells per ml. Traditionally used HEK-cells may grow to cell densities of at the most 10³ cells per ml. It is therefore indeed surprising that the subtypes of HEK 293 cells may grow to densities as high as 10⁹ in suspension under serum-free conditions.

Thus, in an embodiment of the present invention the human host cell may be cultured to a cell density of above 10³ cells per ml, such as above 10⁴ cells per ml, e.g. above 10⁵ cells per ml, such as above 10⁶ cells per ml, e.g. above 10⁷ cells per ml, such as above 10⁸ cells per ml, e.g. above 10⁹ cells per ml, e.g. above 10′⁹ cells per ml, the cell count of which may be further improved, and thereby have a pronounced influence on the yield of protein produced.

The method of purifying the resulting RshTF may be selected from the group consisting of affinity chromatography, ion-exchange chromatography, gel chromatography and any combination thereof. The chromatography may be 2-step chromatography.

In an embodiment of the present invention substantially no synthetic protease inhibitor(s) may need to be added according to the method of the present invention. If synthetic protease inhibitor(s) are added, this is in very small amounts, and to the crude harvested medium comprising the coagulation factor.

Method of Producing RshTF—The Importance of a Correct Glycosylation Pattern

In general, the glycosylation of recombinant proteins produced in transfected cells is inherently derived from the cells in which the recombinant protein is produced. Most proteins undergo post-translational modification (PTM), which can alter their physical and chemical properties, e.g., MW, pI, folding, stability, and biological activity. Glycosylation is the most widely found PTM, with estimations that 80% of all plasma proteins are glycosylated.

Glycosylation is not under direct genetic control, but is dependent on the availability and activity of the various glycosyltransferases, monosaccharides and precursors. Glycosylation of recombinant proteins is dependent on the machinery of the cell line in which they are made.

Thus, the RshTF of the present invention is an extracellular transmembrane glycoprotein which is produced in transfected human cell cultures, i.e. human HEK-cells, Thus, the RshTF of the present invention is also adapting the humanized glycosylation of the RshTF which optimizes its use in humans without causing allergic reactions due to the content of xenogeneic material or xenogeneic glycosylation, which often leads to allergic reaction, for instance when instead of human cells, transfected animal cells are such as Chinese Hamster Ovarian (CHO) or Baby Hamster Kidney cells).

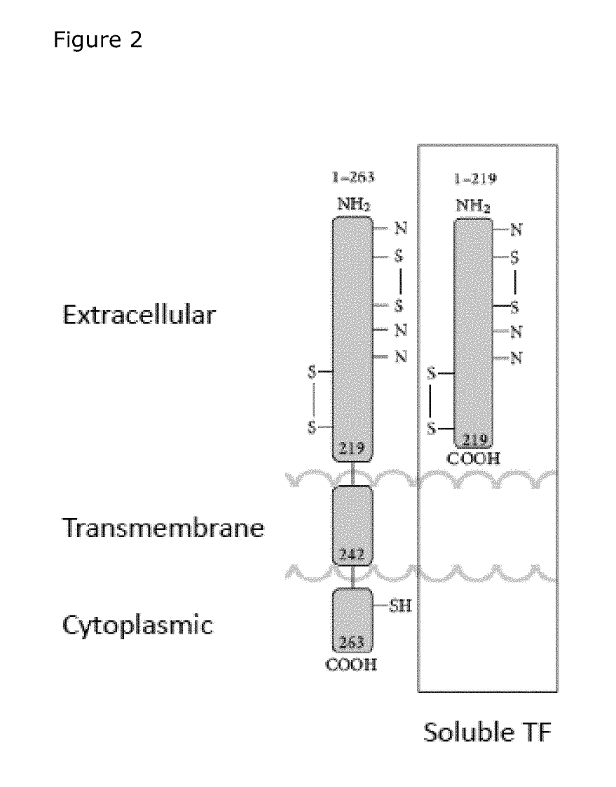

Thus, there are considerable advantages of using the method of the invention when producing RshTF, and especially when producing RshTF from the extracellular domain (SEQ ID NO: 2) as shown in FIG. 2, which represent a schematic diagram of human Tissue Factor.

The RshTF of the present invention is generally produced by transfecting a vector (plasmid) containing at least one cDNA sequence expressing the recombinant Tissue Factor, into host cell, preferably HEK-cells.

These HEK-cells may be sublines of HEK cells such as HEK 293, HEK293T, HEK293E, HEK293 EBNA or PER-C6 cells.

The RshTF-encoding nucleotide sequence (when cloned into a suitable vector) can advantageously be transfected into any of the above-mentioned HEK-cell lines.

Producing RshTF according to the present invention in human HEK-cell lines is particularly advantageous when compared to the conventional techniques of using transfected non-human cell line, such as animal derived cell lines, such as those that have been used for years, such as BHK- and CHO-cells or even less useable in regards to this invention are Human Recombinant Tissue Factor produced in E. Coli.

Because of the heavy glycosylated protein that the RshTF according to the present invention contains in its extracellular domain, it is an advantage that the transfected cell and expression vector system results in the correct human glycosylation both for the sake of the biological behaviour, but also to avoid immune reaction against a foreign, non-human glycosylation pattern when used in man.

Posttranslational modification performed in human cells, influence several physical properties and chemical properties such as glycosylation, phosphorylation, carbocylation, palmitoylation, and neuraminoglycosilation, etc. are of significant importance for the properties of the coagulation factor.

In one embodiment, the RshTF of the present invention is glycosylated with one or more non-reducing disaccharides, preferably trehalose, and/or with one or more disaccharides consisting of sugar-alcohol combinations, preferably mannitol.

In another embodiment, the RshTF of the present invention comprises a glycosylation patterns which is substantially identical to that of the corresponding natural human Tissue Factor.

In still another embodiment, at least 90% of the RshTF of the present invention comprises a glycolysation pattern that results in an immunogenicity response substantially identical to that of the corresponding natural human Tissue Factor.

In still other embodiments, at least 90% of the RshTF of the present invention comprises a glycolysation pattern that results in an immunogenicity response substantially identical to that of the corresponding natural human Tissue Factor, such as at least 91%, at least 92%, least 93%, at least 94%, least 95%, at least 96%, least 97%, at least 98%, least 99%, or 100%, e.g. in the range from 90-100%, such as in the range from 92-99%, such as in the range from 93-98%, such as in the range from 94-97%, such as in the range from 95-96%.

RshTF Formulations—e.g. RshTF Impregnated and/or Coated on Scaffolds

The concentration of RshTF or RshTF comprising protein composition of the present invention for instance impregnated and/or coated on scaffolds is based on results that the inventors found capable of causing blood-clotting effect in a reproducible manner.

When present on a scaffold according to the present invention the concentration of RshTF or RshTF comprising protein composition is in the range from: 0.1 μg-1000 ug per cm² scaffold, preferably 0.2 μg-500 ug per cm² scaffold, more preferably 0.3 μg-250 ug per cm² scaffold, still more preferably 0.4 μg-100 ug per cm² scaffold, still more preferably 0.5 μg-50 ug per cm² scaffold, most preferably 1 μg-25 ug per cm² scaffold.

When present as a solution, the concentration of RshTF or RshTF comprising protein composition is in the range from: 0.1 μg/ml-1000 μg/ml, preferably 0.2 μg/ml-500 μg/ml, more preferably 0.3 μg/ml-250 μg/ml 0.1 μg/ml-1000 μg/ml most preferably 2.5 μg/ml-25 μg/ml.

In a particularly preferred embodiment the concentration of RshTF in solution is in the range from: 2.5 μg/ml.

The concentrations of RshTF or RshTF comprising protein composition on the scaffold depend on the type of scaffold used and whether it will be coated for instance by electro-spraying to scaffolds such as non-resorbable Biatain (Coloplast) or on resorbable gelatin-like scaffolds, or collagen scaffolds, which in this last scaffold in itself can produce some mechanical or physical hemostasis.

Kit-of-Parts: RshTF or RshTF-Containing Protein Compositions used on e.g. a Haemostatic Scaffold

The RshTF of the present invention constitutes as an important part of the Tissue Factor, namely the extracellular domain of the Tissue Factor, which is not an enzyme but a relatively stable glycoprotein with three glycosylation areas.

Instead of using the unstable thrombin as the clotting agent during bleedings, the much more stable RshTF of the present invention, being a novel and reproducible inducer of blood clotting, which is extremely temperature tolerant due to the fact that extracellular part RshTF is very stable, can advantageously be used to quickly and efficiently to treat bleedings in for example a bleeding soldier or patient.

In particular, because of RshTF's binding with an intrinsic factor such as Factor VII/Factor VIIa to form the RshTF:FVII and/or RshTF:FVIIa complexes, which in turn enables this complex/combination to activate Factor X (and Factor X via Factor I X), it would be desirable to impregnate and/or coat for example a haemostatic scaffold with RshTF or protein compositions comprising for example RshTF:FVII and/or RshTF:FVIIa complexes.

Thus, as the RshTF of the present invention is produced as a stable recombinant human glycoprotein, it withstands temperatures significantly better than unstable (labile) intrinsic factors complying much more to what is needed in for example the (battle) field where a clotting protein shall withstand wide range of temperatures.

It is extremely difficult to handle traditional blood clotting products, e.g. in a battlefield where such products typically are carried on/by individuals (such as soldiers in combat, emergency vehicles of any kind).

Thus, in order to provide an easy, quick and safe way of stopping bleedings in for example a bleeding victim or patient, it would be of great importance if for example RshTF or RshTF:FVII and/or RshTF:FVIIa complexes is/are already coated, impregnated or otherwise on the hemostatic carrier (scaffold or alike).

Such products shall for instance when soldiers are in combat be carried in Medipacks and shall be able to withstand harsh conditions and tolerate ambient temperatures, preferably from zero often up to 37° C. without losing its activity.

RshTF or RshTF comprising protein composition of the present invention can be readily coated and/or impregnated onto many types of scaffolds in milligram amounts and carried in for example sealed aluminum foil, easy to tear open and bring in contact with the bleeding with moderate pressure, where it, when applied to the bleeding under pressure will by its binding capacity to intrinsic factors such as Factor VIIa to form RshTF:Factor VIIa start the clotting effect or hemostasis within 7 (seven) minutes.

Thus, because of the stability of the RshTF of the present invention and/or protein composition based thereon when dried, after having been coated onto a scaffold, it can be carried with medical personnel as for instance first aid personnel and can be carried as a scaffold in a medipack and could be used by military personnel who can not bring autolytic products such as thrombin with them because thrombin itself decays within hours to few days without being deep frozen, and is therefore unpractical for use of first aid personnel.

The RshTF or RshTF comprising protein composition of the present invention can be applied alone or together with various carriers and/or stabilisators to a bleeding area, and may bind to zymogen or intrinsic Factor VIIa to form a soluble RshTF:Factor VIIa.

Since the product is essentially serum free it is possible to carry the products in a e.g. a medipack.

A kit-of-parts could contain both RshTF or RshTF comprising protein composition of the present invention, which can be mixed after having been titrated and will most probably be in the area of micrograms to mg/cm² scaffold, e.g. after being dissolved with highly purified recombinant prothrombin or recombinant preprothrombin at an amount of 140 IU/cm scaffold (or recombinant prethrombin).

In addition, in said kit-of-parts RshTF or RshTF comprising protein composition of the present invention either alone or in combination with recombinant prothrombin, recombinant preprothrombin or recombinant prothrombin can be added to a carrier as for instance biatain (Coloplast) or MPEG PLGA (Coloplast).

In another embodiment, in the case of MPEG-PLGA scaffolds, such a scaffold could be mixed with a paste that can be applied a cream applied directly on bleedings known in the art as “sieving of blood” from e.g. a surgical area.

Unlike thrombin the RshTF or RshTF comprising protein composition of the present invention have several month of stability especially if the recombinant proteins are dried after being coated to the synthetic membrane or other membranes and can be stored most probably up to between 0.5 to 1 year, e.g. when packed as a kit.

The scaffold comprising RshTF or RshTF comprising protein composition of the present invention can be applied to the bleeding area, by adding some pressure to facilitate hemostasis. The hemostasis is effected by the mixing of RshTF or RshTF comprising protein with endogenous clotting factors such as Factor VII/VIIa, which through Factor IX activates Factor X to Factor Xa.

In an embodiment, the RshTF or RshTF comprising protein composition of the present invention may be in form of a powder (such as but not limited to MPEG PLGA+CaCl₂), a gel or present in a scaffold.

In a further embodiment, such a scaffold may e.g. be Biatain, which is polyurethane foam, or alternative as collagen or gelatin derived paste, that can be applied in using various application methods including via a syringe system or spray system.

An embodiment of the invention pertains to a kit-of-parts comprising:
(i) A scaffold which may e.g. be Biatain, which is polyurethane foam, or alternative as collagen or gelatin derived paste, that can be applied in using various application methods including via a syringe system or spray system, comprising the RshTF or RshTF comprising protein composition of the present invention.

Another embodiment pertains to a kit-of-parts comprising:
(i) A gel or granulate or a gelatin, or a paste comprising the RshTF or RshTF comprising protein composition of the present invention.

Another embodiment pertains to a kit-of-parts comprising:
(i) a scaffold comprising the RshTF or RshTF comprising protein composition of the present invention and
(ii) recombinant prothrombin (or preprothrombin)

Another embodiment pertains to a kit-of-parts comprising:
(i) a gel or granulate or a gelatin comprising RshTF or RshTF comprising protein composition of the present invention and
(ii) recombinant prothrombin (preprothrombin)

Also envisaged is a “IA RshTF Kit” prepared as a “prophylactic” kit, capable of securing hemostasis, after the surgeon has completed the surgery, but is unsure of, whether post operative bleeding may occur. This is also be envisioned to happen, when a dentist or a dental surgeon needs postoperative security for bleeding.

This so-called “Prophylactic hemostasis Kit” will ensure that the bleedings or sieving of bleedings, that relatively often happens in the postoperative phase, can be treated by applying a prophylactic combination of RshTF or RshTF comprising protein composition of the present invention for instance in a paste, added to another area, where the surgeon may anticipate a postoperative bleeding, so instead of using application of kits combining thrombin and fibrinogen (such as Baxter's Tiseel glue), where one could have a spray or a paste to apply to an area, that may be expected to be capable of causing post operative bleedings.

For instance during orthopaedic surgery in bone structures, where it is extremely difficult to apply suturing, a paste or a spray containing RshTF or RshTF comprising protein composition of the present invention can be applied, when the surgeon is closing the operated area, and has to rely on the fact that everything has been done to avoid postoperative bleedings. Likewise, neurosurgeons and dentists are often in need of such products.

The same is true for cardiovascular surgeons. In such cases, it is not sufficient at all to apply thrombin in such prophylactic kit; and there is often no need to use combinations of thrombin and fibrinogen mix, such as Tiseel glue, etc. where one instead can apply a glycoprotein such as RshTF in a carrier such as paste, foam, or may be even thin layers of membrane's coated with RshTF or RshTF comprising protein composition of the present invention.

In another aspect of the invention, it is anticipated that RshTF or RshTF comprising protein composition of the present invention can be mixed with either prothrombin and/or thrombin (of the type of thrombin that HumaCell (Soon Jeong, HumanCell, Naperville, Ill., USA) as this may improve the stability when compared to all other types of recombinant human thrombin.

One or more of these improved recombinant intrinsic factors, which is proprietary to HumanCell, could accelerate the clotting or hemostasis effect to in the area of seconds (e.g., 12-200 seconds), where it is anticipated that even though when RshTF is applied in vivo in animals or humans (topical application), the clotting time may shorten—also when using a scaffold such as special types of collagen, membranes, or paste, in which RshTF or RshTF comprising protein composition of the present invention can be mixed from the factory, and distributed for instance with minimal humidity or even when filled into a sealed bag, using N₂ gas during filling to obtain as low humidity as possible, or even by adding humidity absorbing salts.

The ideal matrix for hemostatic treatment has to be capable of adhering to any mammal tissue surfaces including bleeding surfaces to prevent bleeding during e.g. surgery and to conserve the patient's own blood volume, or at least minimize the necessity for the usage of natural blood products on the patient. RshTF or RshTF comprising protein composition of the present invention in a kit with or without added recombinant prothrombin or prothrombin, added to a bleeding surface will form a clot and work as a hemostatic.

In an embodiment, the scaffold may be selected from the group consisting of a dressing, a patch, a sponge, such as Tacosil, Hema-Seal, Biatain, MPEG-PLGA , Aseed, Spongostan and Surgifoamm Surgiflo, other gelatin like products or collagen type products, from for example of porcine origin.

In a further embodiment, the scaffold matrix can also constitute a paste, which can be distributed over a blood sieving area, in which it is difficult or impossible to place sutures, then the paste can be covering the area, where the surgeon has performed his surgery on the patient, and then work as a haemostat product after surgery has been completed, and in this manner the surgeon can ensure him/her self, that if sieve bleeding should occur after he/she has closed the layers over the surgical field. The past containing RshTF or RshTF comprising protein composition of the present invention will just stay there most probably for some time (minutes to hours) before resorption, and if bleeding should start after the fact that the surgeon has closed the area and found that it is not bleeding, any bleeding occurring afterwards, for instance in relative close connection to the postoperative period (minutes to hours), the RshTF or RshTF comprising protein composition of the present invention will do nothing before, the patient might start bleeding again postoperatively, and the intrinsic factors via Factor VII /VIIa will then form activation of Factor IX and/or Factor X, and thrombin conversion will occur and clotting be the result.

This prophylactic treatment makes use of RshTF or RshTF comprising protein composition of the present invention embedded in a carrier placed on the operation area, which will not be activated post operatively before, a possible bleeding should occur, after the wound has been closed or the operation area has been closed.

The amount of the RshTF or RshTF comprising protein composition of the present invention may vary depending on the intended use of the product, the more severe bleeding, the higher a concentration of the one or more coagulation factor is needed. Suitably, the amount of the RshTF or RshTF comprising protein composition of the present invention in microgram up to mg/cm² and recombinant prothrombin (or preprothrombin) is in the range from 70 -1000 to 5000 U/cm² or a comparable measured amount taking into account the measurement cm². It is envisaged that an amount of 10-1000 U/cm² may be suitable in a device for stopping severe bleedings.

When applying the RshTF or RshTF comprising protein composition of the present invention, there can be difference in time to hemostasis, and some products such as porcine collagen derived products may add to the hemostasis, i.e. besides the “classical” hemostasis that is normally caused by activation of the intrinsic zymogens to activated enzymes, ending with activated thrombin that again will convert soluble fibrinogen to insoluble fibrin. The clotting efficacy also include the solidity of a clotting that can be accelerated of factors already present in the blood such as for instance platelets.

Therefore, two (2) variations of this Hemostatic kit may be envisaged, namely a kit comprising of RshTF (the extra cellular part) or RshTF comprising protein composition of the present invention.

In cases where a bleeding patient may suffer from bleedings and be low in prothrombin, another Hemostatic kit for these types of patients will contain both RshTF (the extra cellular part) or RshTF comprising protein at an amount of 2.5μg—or even 1 μg/mL or e.g., 2.5 μg or even 1 μg/cm² scaffold, together with 70 to 150 units of prothrombin, corresponding to 10-40 μg/cm². The recombinant human prothrombin or preprothrombin, which will be the most useable protein to be mixed together with the RshTF (the extra cellular part) or RshTF comprising protein (whereas thrombin (Factor IIa) will not be capable of being used for this purpose).

Enhanced Stability of the RshTF by Using Dissacharides and/or Polysarcharides

The stability of RshTF glycoprotein can be enhanced by dissacharides, such as for instance isomalt or mannitol. Isomalt or mannitol is/are a sugar substitute, a type of sugar alcohol, used primarily for its sugar-like physical properties. Isomalt or mannitol will, when added to a glycoprotein such as RshTF in the same amount (weight to weight) as the amount of RshTF, stabilize the RshTF. Isomalt or mannitol is an equimolar mixture of two disaccharides, each composed of two sugars: glucose and mannitol (α-D-glucopyranosido-1,6-mannitol), also glucose and sorbitol (α-D-glucopyranosido-1,6-sorbitol).

In this manner one can add a certain amount of non-reducing types of sugar components such as non-reducing disaccharides, which could be any non-reducing disaccharides such as e.g. trehalose, or disaccharides consisting of sugar-alcohol combinations such as e.g. isomalt or mannitol. Furthermore, larger molecule such as dextran could be used, because it is also a no-reducing sugar combination.

Complete hydrolysis of isomalt or mannitol yields glucose (50%), sorbitol (25%), and mannitol (25%).

Other disaccharides could be used instead of isomalt or mannitol, but mannitol is the preferred dissacharide that can be used as stabilisator. The common sugar alcohols, maltitol, erythritol, and hydrogenated starch hydrolysates, are manufactured from cornstarch. Xylitol, another common sugar alcohol, is manufactured from such sources as corncobs, sugar cane bagasse (stalk residue remaining after sugar extraction), or birch wood waste. Isomalt or mannitol and lactitol are becoming more common and are manufactured from sucrose and whey, respectively.

Lyophilization of RshTF

When lyophilizing RshTF, maltose and Tween are added in order to obtain an easily soluble freeze-dried powder to avoid a freeze-dried product not easily resolved in solvents, which often would be PBS at pH 6.5 to 7.5. The RshTF is freeze-dried in adequate portions, easy for the user to re-dissolve and apply to a carrier or to create a spray form of RshTF. Instead of the combination of maltose and Tween, one can use mannitol together with Tween.

RshTF's Dependency on the Intrinsic Factor VII/VIIa by in Order to Induce and Accelerate Hemostasis

For a more detailed description of the blood cascade reactions starting, see FIG. 1.

Thus, as depicted in FIG. 1, after vascular injury, clotting is initiated by the binding of plasma FVII/FVIIa to Tissue Factor (TF) (also known in the art as coagulation Factor III or tissue thromboplastin). The TF:FVII/VIIa complex of the extrinsic pathway initiates blood coagulation by activating both FX and FIX. The FVIIIa:FIXa complex of the intrinsic pathway provides an alternative route to generate FXa, which participates in the prothrombinase complex (FVa:FXa). This complex activates prothrombin to thrombin, which plays a central role in the coagulation

During bleeding the source of Tissue Factor is released from endothelial cells which are damaged or torn apart because of damage to blood vessels. The released endogenous Tissue Factor (Factor III) are then responsible for binding to Factor VII/Factor VIIa, to form TF:VIIa. The amount of Tissue Factor binding to Factor VIIa to form TF:Factor VIIa is most probably not sufficient to prevent larger bleeding.

Under those circumstances, when Factor VII/VIIa (such as Novo Nordisk NovoSeven) is given parenterally, there may not be sufficient amount of Tissue Factor to form sufficient amount of Factor VIIa (NovoSeven) to activate the cascade (see FIG. 1) to end up generating sufficient amount of Factor Xa and thus no sufficient amount of activated Factor IIa, also called thrombin is present to convert soluble fibrinogen to insoluble fibrin.

In this context, RshTF or RshTF comprising protein composition of the present invention has proven to activate prothrombin to thrombin as evidenced by PPP (platelet-poor plasma) Thrombography (see e.g. example 10) thus providing evidence of the fact that the RshTF of the present invention (produced by using transfected human HEK-cells to produce the extracellular part (SEQ ID NO: 2) of the Tissue Factor) and capable of reacting through binding of RshTF to Factor VII/VIIa to form RshTF:FVIIa (RgsTF:FVIIa) by exposing platelet poor plasma by solely exposing the platelet poor plasma to the RshTF.

One of the reasons why Factor VII/Factor VIIa has been able to cause induction and acceleration of activation of thrombin to convert soluble fibrinogen to insoluble fibrin in the blood coagulation cascade, is partly caused by insufficient endogenous presence of Tissue Factor or soluble Tissue Factor capable of binding to the injected Factor VII/Factor VIIa to form sufficient amount of TF:FVIIa complex to induce Factor Xa (or via Factor IXa to induce Factor Xa), which in turn produces sufficient amount of thrombin to be converted into fibrinogen. This is one of the reasons claimed to be the cause of the effect by administering systemic Factor VIIa alone (such as NovoSeven).

By combining RshTF or RshTF comprising protein composition of the present invention topically to the bleeding area, at the same time as systemically administering Factor VII/VIIa (such as NovoSeven) into the patient, the topically applied RshTF or RshTF comprising protein composition of the present invention will together with the Factor VII/FVIIa be capable of more efficiently stop the bleeding at the bleeding site.

Should the amount of prothrombin in the bleeding area be too small, recombinant prothrombin may be administered together with the topical application of RshTF or RshTF comprising protein composition of the present invention to provide sufficient supply of prothrombin to be converted to thrombin and thereby adding to the hemostasis effect.

In case where applied Factor VII/VIIa is given to trauma patients with severe bleedings, Factor VIIa may not be able to connect with sufficient amount of Tissue Factor to activate Factor Xa. Therefore the addition of RshTF or RshTF comprising protein composition of the present invention (applied topically) to the bleeding area, may utilize the systemically injected recombinant Factor VIIa to induce activation of Factor Xa and activate prothrombin to produce activated Factor Ha (thrombin), to create sufficient hemostasis within the limited time period, where the patient has not yet reached a phase, where the bleeding has been too large to prevent using this approach.

Protein Composition Comprising RshTF in Combination with e.g. One or More Addition Haemostatic Agent(s)

If it should appear that a severe bleeding would be stopped faster, by adding a surplus of other factors, one intrinsic factor that might be added systemically would be recombinant Factor VII/ VIIa which would be bound to RshTF or RshTF comprising protein composition of the present invention to form RshTF:FVIIa or RshTF:VII/VIIa to further activate the zymogens Factor X to Xa and ending up activating zymogen Factor II, prothrombin to activated Factor II, thrombin which would convert soluble fibrinogen to insoluble fibrin, and together with released platelets among others, would form a solid clot.

The ideal product for stopping excessive bleedings may be Tissue Factor or rather RshTF or RshTF comprising protein composition of the present invention, which as the extrinsic factor would act together with Factor VII (Factor VIIa would form TF:FVIIa, etc.). As described above, one could also ensure an accelerated hemostasis by adding prothombin or a thrombin (e.g. from HumanCell, Naperville, Ill.).

Preprothrombin is defined as a two chain, disulphide-bonded, glycosylated polypeptide that when activated to thrombin cleaves specific bonds in fibrinogen to produce fibrin monomers that self-assemble to form a fibrin clot, when activated to thrombin.

When mixing preprothrombin, activated, with RshTF or RshTF comprising protein composition of the present invention, to thrombin together with fibrinogen, the final stage of the natural fibrin clotting cascade takes place. In addition to providing hemostasis, this fibrin sealant also provides ideal environment for fibroblastic proliferation as well as proliferation of other mesenchymal cells.

In an embodiment of the invention, the recombinant human prothrombin to be combined with RshTF or RshTF comprising protein composition of the present invention is a preprothombin.

The RshTF or RshTF comprising protein composition of the present invention administered topically as for instance on a carrier or scaffold that again will activate Factor X to Factor Xa. Then Factor Xa will activate either endogenous prothrombin present in the bleeding area, or applied preprothrombin (for instance as a freeze-dried mix of recombinant Tissue Factor and preprothrombin supplied on a carrier or scaffold and kept in a sealed package, and opened at the time of the bleeding being treated).

In this manner the resulting activated thrombin will either be activated from endogenous prothrombin or from exogenous preprothrombin. This activation of thrombin will start converting fibrinogen to fibrin and thereby, efficient and fast clotting is obtained.

Modes of Co-Administration

In an embodiment of the invention, the RshTF is for use in topical treatment and/or prevention of bleedings wherein said recombinant soluble human Tissue Factor is administered together with soluble fibrinogen as a co-administration. When the RshTF is administered topically via e.g. a scaffold, the soluble fibrinogen can be administered intravenously (IV) or topically, e.g. admixed in the scaffold together with the RshTF

This co-administration accelerate conversion of soluble made available either by systemic application by intravenous or other routes for the organism to have sufficient amount of fibrinogen in his/her blood to enable conversion of sufficient amount of soluble fibrinogen to insoluble fibrin.

Outline of Subsequent Experimental Tests

After production and purification of the RshTF, thrombography experiments were performed to determine the functional activity of RshTF (performed at the Hematology Laboratory at Washington University Medical School in St. Louis (Mo., USA)).

In addition, the inventors performed two functional activity assays with fresh human blood: thrombin generation in platelet poor plasma (PPP) and clot formation in whole blood. Both assays proved that RshTF alone triggered thrombin generation in PPP without exogenous phospholipids and formed a clot in whole blood in a shorter time. These encouraging findings justify homeostatic validation in animal models and clinical testing as a kit containing a carrier on to which RshTF of the present invention is coated, impregnated or otherwise applied.

Thus, in the above-context, an object of the present invention relates to the provision of a stable blood clotting agent which tolerates ambient temperatures without losing activity, is easy to produce, can be produced in high yields and having low antigenicity when used to treat and/or prevent bleedings in humans.

In particular, it is an object of the present invention to provide a RshTF comprising the SEQ ID NO: 2, wherein the RshTF is produced in a human cell, that solves the above mentioned problems of the prior art in relation to traditionally used blood clotting agents.

Thus, one aspect of the invention relates to a RshTF comprising the SEQ ID NO: 2, wherein said recombinant soluble human Tissue Factor is produced in a human cell.

Another aspect of the present invention relates to a protein composition comprising RshTF in combination with one or more intrinsic factor, such as e.g. Factor VII/VIIa, participating in blood clotting or hemostasis.

Yet another aspect of the present invention is to provide a kit-of parts comprising an essentially air- and humidity-tight receptacle comprising a scaffold impregnated and/or coated with RshTF or a protein composition comprising RshTF, and optionally a humidity absorbing material.

Still another aspect of the present invention is to provide a method of producing recombinant soluble human Tissue Factor, said method comprising culturing in vitro a human host cell transfected with at least one exogenous nucleic acid sequence encoding said recombinant soluble human Tissue Factor, wherein the human host cell is a human HEK cell.

Items

Item 1: A recombinant soluble human Tissue Factor (RshTF) comprising the SEQ ID NO: 2, wherein said recombinant soluble human Tissue Factor is produced in a human cell.

Item 2: The recombinant soluble human Tissue Factor according to item 1, wherein the human cell is a human HEK cell selected from the group consisting of human HEK 293 cells, human HEK293T cells, human HEK293E cells or human HEK293 cells.

Item 3: The recombinant soluble human Tissue Factor according to any of items 1-2, wherein the recombinant soluble human Tissue Factor is essentially serum-free.

Item 4: The recombinant soluble human Tissue Factor according to any of items 1-3, wherein the recombinant soluble human Tissue Factor is glycosylated with one or more non-reducing disaccharides, preferably trehalose, and/or with one or more disaccharides consisting of sugar-alcohol combinations, preferably mannitol.

Item 5: The recombinant soluble human Tissue Factor according to any of items 1-4, wherein at least 90% of the recombinant soluble human Tissue Factor comprises a glycolysation pattern that results in an immunogenicity response substantially identical to that of the corresponding natural human Tissue Factor.

Item 6: The recombinant soluble human Tissue Factor according to any of items 1-5, wherein said recombinant soluble human Tissue Factor is part of a glue, a powder, a spray, a gel, a granulate, a gelatin, a paste, a fluid, a suture, a scaffold, a paste and/or a cream substance to which said Factor can be bound or mixed.

Item 7: The recombinant soluble human Tissue Factor according to item 6, wherein the powder is MPEG PLGA.

Item 8: The recombinant soluble human Tissue Factor according to item 6, wherein the scaffold is a haemostatic scaffold in the form of a gel, a foam, polyurethane foam, a gelatin-like substance, a collagen, a collagen-derived paste and/or a gelatin-derived paste.

Item 9: The recombinant soluble human Tissue Factor according to item 8, wherein the concentration of recombinant soluble human Tissue Factor on the scaffold range from 0.1 μg-1000 ug per cm² scaffold.

Item 10: The recombinant soluble human Tissue Factor according to any of items 1-5, wherein the recombinant soluble human Tissue Factor is in a solution in a concentration in the range from 0.1 μg/ml-1000 μg/ml.

Item 11: The recombinant soluble human Tissue Factor according to any of items 1-10, for use in topical treatment and/or prevention of bleedings.

Item 12: The recombinant soluble human Tissue Factor according to any of items 1-10, for topical use as a tissue sealant and/or to facilitate tissue adherence.

Item 13: The recombinant soluble human Tissue Factor according to any of items 1-10, for use in topical treatment and/or prevention of bleedings wherein said recombinant soluble human Tissue Factor is administered together with soluble fibrinogen as a co-administration.

Item 14: The recombinant soluble human Tissue Factor according to any of items 1-10, for use in topical treatment and/or prevention of bleedings wherein said recombinant soluble human Tissue Factor is administered concomitantly with one or more systemic administration(s) of Factor VII and/or Factor VIIa.

Item 15: The recombinant soluble human Tissue Factor according to any of items 1-10, for use in topical treatment and/or prevention of bleedings wherein said recombinant soluble human Tissue Factor is administered concomitantly with adding collagen to the bleeding site.

Item 16: A protein composition comprising recombinant soluble human Tissue Factor according to any of items 1-5, in combination with one or more agents selected from the group consisting of: fibrinogen (Factor I), prothrombin (Factor II), any other composition of prothrombin such as for instance preprothrombin and prethrombin, thrombin, Tissue Factor (Factor III), Labile Factor (Factor V), proconvertin (Factor VII), Factor VII/VIIa (NovoSeven), Antihaemophilic Factor (Factor VIII), Christmas Factor (Factor IX), Stuart-Prower Factor (Factor X), Plasma thromboplastic (Factor XI), Hageman Factor (Factor XII), Factor XIII, Fibrin-stabilizing Factor (transamidase or Factor XIIIa), von Willebrand Factor, Prekallikrein (Fletcher Factor) high-molecular-weight kininogen (HMWK), fibronectin or any intrinsic Factor participating in blood clotting or hemostasis.

Item 17: The protein composition according to item 16, wherein said protein composition is part of a glue, a powder, a spray, a gel, a granulate, a gelatin, a paste, a fluid, a suture, a scaffold, a paste and/or a cream substance to which said Factor can be bound or mixed.

Item 18: The protein composition according to item 17, wherein the powder is MPEG PLGA.

Item 19: The protein composition according to item 17, wherein the scaffold is a haemostatic scaffold in the form of a gel, a foam, polyurethane foam, a gelatin-like substance, a collagen, a collagen-derived paste and/or a gelatin-derived paste.

Item 20: The protein composition according to item 19, wherein the concentration of protein composition on the scaffold range from 0.1 μg-1000 ug per cm² scaffold.

Item 21: The protein composition according to item 16, wherein the protein composition is in a solution in a concentration in the range from 0.1 μg/ml-1000 μg/ml.

Item 22: The protein composition according to any of items 16-21, for use in topical treatment and/or prevention of bleedings.

Item 23: The protein composition according to any of items 16-21, for topical use as a tissue sealant and/or to facilitate tissue adherence.

Item 24: The protein composition according to any of items 16-21, for use in topical treatment and/or prevention of bleedings wherein said protein composition is administered together with soluble fibrinogen as a co-administration.

Item 25: The protein composition according to any of items 16-21, for use in topical treatment and/or prevention of bleedings wherein said protein composition is administered concomitantly with one or more systemic administration(s) of Factor VII and/or Factor VIIa.

Item 26: The protein composition according to any of items 16-21, for use in topical treatment and/or prevention of bleedings wherein said protein composition is administered concomitantly with adding collagen to the bleeding site.

Item 27: A kit-of parts comprising an essentially air- and humidity-tight receptacle comprising a scaffold impregnated and/or coated with recombinant soluble human Tissue Factor according to any of items 1-5, 8-9 or a protein composition according to any of items 16, 19-20, and optionally a humidity absorbing material.

Item 28: Method of treating and/or preventing bleedings in humans comprising topical administration on the bleeding site in a human with a recombinant soluble human Tissue Factor according to any of items 1-10 and/or one or more of the protein compositions according to any of items 16-21.

Item 29: A method of producing recombinant soluble human Tissue Factor (RshTF), said method comprising culturing in vitro a human host cell transfected with at least one exogenous nucleic acid sequence encoding said recombinant soluble human Tissue Factor, wherein the human host cell is a human HEK cell.

Item 30: The method according to item 29, wherein the human HEK cell is selected from the group consisting of human HEK 293 cells, human HEK293T cells , human HEK293E cells or human HEK293 cells.

Item 31: The method according to any of items 29-30, wherein the in vitro culturing is carried out under essentially serum-free conditions.

Item 32: The method according to any of items 29-31, wherein the transfected human HEK cell is cultured to a cell density between 3-9 million cells/mL culture medium.

Item 33: The method according to any of items 29-32, wherein the recombinant soluble human Tissue Factor is essentially serum-free.

Item 34: The method according to any of items 29-33, wherein the exogenous nucleic acid sequence comprises SEQ ID NO: 1.

Item 35: A recombinant soluble human Tissue Factor obtainable by a method according to any of items 29-34.

Item 36: The recombinant soluble human Tissue Factor according to item 35, wherein the recombinant soluble human Tissue Factor is glycosylated with one or more non-reducing disaccharides, preferably trehalose, and/or with one or more disaccharides consisting of sugar-alcohol combinations, preferably mannitol.

Item 37: The recombinant soluble human Tissue Factor according to item 36, wherein at least 90% of the recombinant soluble human Tissue Factor comprises a glycolysation pattern that results in an immunogenicity response substantially identical to that of the corresponding natural human Tissue Factor.

Item 38: The recombinant soluble human Tissue Factor according to any of items 35-37, wherein said recombinant soluble human Tissue Factor is part of a glue, a powder, a spray, a gel, a granulate, a gelatin, a paste, a fluid, a suture, a scaffold, a paste and/or a cream substance to which said Factor can be bound or mixed.

Item 39: The recombinant soluble human Tissue Factor according to item 38, wherein the powder is MPEG PLGA.

Item 40: The recombinant soluble human Tissue Factor according to item 38, wherein the scaffold is a haemostatic scaffold in the form of a gel, a foam, polyurethane foam, a gelatin-like substance, a collagen, a collagen-derived paste and/or a gelatin-derived paste.

Item 41: The recombinant soluble human Tissue Factor according to item 40, wherein the concentration of recombinant soluble human Tissue Factor on the scaffold range from 0.1 μg-1000 ug per cm² scaffold.

Item 42: The recombinant soluble human Tissue Factor according to item 35-37, wherein the recombinant soluble human Tissue Factor is in a solution in a concentration in the range from 0.1 μg/ml-1000 μg/ml.

Item 43: The recombinant soluble human Tissue Factor according to any of items 35-42, for use in topical treatment and/or prevention of bleedings.

Item 44: The recombinant soluble human Tissue Factor according to any of items 35-42, for topical use as a tissue sealant and/or to facilitate tissue adherence.

Item 45: The recombinant soluble human Tissue Factor according to any of items 35-42, for use in topical treatment and/or prevention of bleedings wherein said protein composition is administered together with soluble fibrinogen as a co-administration.

Item 46: The recombinant soluble human Tissue Factor according to any of items 35-42, for use in topical treatment and/or prevention of bleedings wherein said protein composition is administered concomitantly with one or more systemic administration(s) of Factor VII and/or Factor VIIa.

Item 47: The recombinant soluble human Tissue Factor according to any of items 35-42, for use in topical treatment and/or prevention of bleedings wherein said protein composition is administered concomitantly with adding collagen to the bleeding site.

Item 48: The recombinant soluble human Tissue Factor according to any of items 35-42, for use in topical treatment and/or prevention of bleedings wherein said recombinant soluble human Tissue Factor is administered together with soluble fibrinogen as a co-administration.

Item 49: A protein composition comprising recombinant soluble human Tissue Factor obtainable by a method according to any of items 29-34, in combination with one or more agents selected from the group consisting of: fibrinogen (Factor I), prothrombin (Factor II), any other composition of prothrombin such as for instance preprothrombin and prethrombin, thrombin, Tissue Factor (Factor III), Labile Factor (Factor V), proconvertin (Factor VII), Factor VII/VIIa (NovoSeven), Antihaemophilic Factor (Factor VIII), Christmas Factor (Factor IX), Stuart-Prower Factor (Factor X), Plasma thromboplastic (Factor XI), Hageman Factor (Factor XII), Factor XIII, Fibrin-stabilizing Factor (transamidase or Factor XIIIa), von Willebrand Factor, Prekallikrein (Fletcher Factor) high-molecular-weight kininogen (HMWK), fibronectin or any intrinsic Factor participating in blood clotting or hemostasis.

Item 50: The protein composition according to item 49, wherein said protein composition is part of a glue, a powder, a spray, a gel, a granulate, a gelatin, a paste, a fluid, a suture, a scaffold, a paste and/or a cream substance to which said Factor can be bound or mixed.

Item 51: The protein composition according to item 50, wherein the powder is MPEG PLGA.

Item 52: The protein composition according to item 50, wherein the scaffold is a haemostatic scaffold in the form of a gel, a foam, polyurethane foam, a gelatin-like substance, a collagen, a collagen-derived paste and/or a gelatin-derived paste.

Item 53: The protein composition according to item 52, wherein the concentration of protein composition on the scaffold range from 0.1 μg-1000 ug per cm² scaffold.

Item 54: The protein composition according to item 49, wherein the protein composition is in a solution in a concentration in the range from 0.1 μg/ml-1000 μg/ml.

Item 55: The protein composition according to any of items 49-54, for use in topical treatment and/or prevention of bleedings.

Item 56: The protein composition according to any of items 49-54, for topical use as a tissue sealant and/or to facilitate tissue adherence.

Item 57: The protein composition according to any of items 49-54, for use in topical treatment and/or prevention of bleedings wherein said recombinant soluble human Tissue Factor is administered together with soluble fibrinogen as a co-administration.

Item 58: A kit-of parts comprising an essentially air- and humidity-tight receptacle comprising a scaffold impregnated and/or coated with recombinant soluble human Tissue Factor obtainable by a method according to any of items 29-34 or a scaffold according to item 40-41, and a protein composition according to any of items 49-54, and optionally a humidity absorbing material.

Item 59: Method of treating and/or preventing bleedings comprising topical administration on the bleeding site in a human with a recombinant soluble human Tissue Factor obtainable by a method according to any of items 29-34 or recombinant soluble human Tissue Factor according to any of items 35-42 and/or one or more of the protein compositions according to any of items 49-54.

The invention will now be described in further details in the following non-limiting examples.

### EXAMPLES

In the below examples the following tools, media, apparatus etc. have been applied (table 2).

TABLE 2

| Description | Supplier | Cat No. |
|---|---|---|
| DMEM | LifeTecnologies | 11960 |
| L-Glutamine, 200 mM | LifeTecnologies | 25030 |
| 1M HEPES | LifeTecnologies | 15630 |
| MEM NEAA 100X | LifeTecnologies | 11140 |
| PBS | LifeTecnologies | 10010 |
| TrypLE | LifeTecnologies | 12605 |
| CD293 | LifeTecnologies | 11913 |
| Zeocin | LifeTecnologies | R250-05 |
| NuPAGE (SDS-PAGE) | LifeTecnologies | NP0322BOX |
| 100 mm Petri Dish | MIdSci | TP93100 |
| 125 mL Erlenmeyer flask | VWR | 89095-258 |
| 250 mL Erlenmeyer flask | VWR | 89095-268 |
| 2 L Erlenmeyer flask | VWR | 10126-438 |
| HiTrap ANX FF | GE Life Sciences | 17-5163-01 |
| HiTrap Heparin HP | GE Life Sciences | 17-0407-03 |
| HiTrap Q FF | GE Life Sciences | 17-5156-01 |
| Thrombin calibrator | Stago | 86192 |
| FluCa Kit | Stago | 86197 |

### Example 1

Method of Producing RshTF—Overview (Table 3)

TABLE 3

| Culture medium & | Liquid CD 293: Invitrogen (#11913) | |
|---|---|---|
| supplements | 200 mM L-glutamine: Invitrogen (#25030) | |
| | 1M HEPES: Invitrogen (#15630) | |
| | Zeocin ™ (100 mg/mL): Invitrogen (#R250) | |
| | Suspension culture medium: CD 293 (1 L) + | |
| | 4 mM L-glutamine + 25 mM HEPES at final | |
| | concentrations | |
| | Zecoin medium: Suspension culture medium + | |
| | 50 ug/mL zeocin at final concentration (CAN | |
| | BE OMITTED for large scale production) | |
| Thaw & Seeding | | |
| [symbol] | 1. | Thaw 1 vial of each cell line in 37° C. |
| | | water bath |
| | 2. | After mixing by pipetting 3-4 times the |
| | | cells to be transferred into 10 ml of |
| | | suspension culture medium in 125 mL |
| | | Erlenmeyer flask with vent cap (VWR, |
| | | #30180-036) |
| | 3. | Suspension culture the cells at 125 rpm |
| | | overnight |
| Growing | | |
| [symbol] | 1. | On day 2 count the viable cell density |
| | | (VCD) and adjust VCD to one million |
| | | cells/mL with 50 ug/mL zeocin supplied |
| | | suspension medium |
| | 2. | On day 3 and/or day 4 count VCD and |
| | | adjust to 1M/mL until reach 50 mL culture |
| | 3. | Once reach 50 mL culture with NLT 90% |
| | | viability adjust VCD to 0.5M/mL with the |
| | | fresh zeocin medium |
| | 4. | Grow the cells for 7 days in the zeocin |
| | | medium |
| Scale-up process | | |
| [symbol] | 1. | At day 7 count VCD and adjust to |
| | | 0.5M/mL with the fresh zeocin medium: |
| | | 125 mL flask (VWR, #30180-036, |
| | | up to 50 mL culture), 250 mL flask |
| | | (VWR, #30180-044, up to 100 mL |
| | | culture), or 2 L flask (VWR, |
| | | #83014-018, up to 800 mL culture) |
| | 2. | Grow for 7 days between the passages |
| | 3. | Once culture volume reaches 800 mL |
| | | culture next larger scale requires spinner |
| | | or bioreactor. Each passage yields four |
| | | times larger volume culture. |
| Cell harvest | | Cells for passage to be harvested by |
| | | centrifugation at 200 × g for 5 min. |
| Spent medium | | Recombinant protein spent medium to be |
| harvest | | harvested by centrifugation 10,000 × g for |
| | | 20 min and stored at −20° C. until performing |
| | | purification All centrifuge tubes/bottles to be |
| | | pyrogen free and autoclaved. Spent medium |
| | | must be handled in a bio-safety cabinet to |
| | | avoid introducing endotoxin |

HEK293TS cell line, a serum-free and suspension adapted cell line, was generated from HEK293T (original HEK293T cell line used for transfection of the gene for extracellular soluble human Tissue Factor is from HumanCell Inc., Naperville, Ill. 60540, purchased from GenHunter (TN, USA)). Cell culture media, supplements, and zeocin were purchased from LifeTechnologies (CA, USA). Bovine calf serum was purchased from HyClone (UT, USA). Plastic culture dishes were from MidSci (MO, USA), and Erlenmeyer culture flasks from VWR (PA, USA). Protein purification prepacked columns were from GE Lifesciences (NJ, USA). Thrombin generation assay reagents were from Diagnostica Stago (NJ, USA). Detailed provider information is in Table 2.

The production of RshTF was split up into 3 steps, where step 1 consists of obtaining the cDNA of hTF (BC011029) and construct the expression plasmid encoding the Tissue Factor protein, 1-219 amino acid residues. Then the coding sequence was confirmed to be authentic. The construct was transfected into HEK 293T cells and expression was confirmed.

Step 2 consisted of selecting the cell lines that appeared to be stably transfected. The transfected HEK293T cell line was adapted to suspension culture and further adapted to serum- free media culturing. The transfected HEK293T cell culture in suspension was then expanded in a one (1) litre with a cell density of approximately 3-3.5 million cells/mL. The third (3rd) step consisted of purification by column chromatography as shown in the following table 4.

TABLE 4

| Activities | | Duration | |
|---|---|---|---|
| Step 1: | | 4-5 | weeks |
| a) | Obtain cDNA of hTF (BC011029) and construct | | |
| | expression plasmid encoding the protein of 1-219 | | |
| | amino acid residues (2-3 weeks) | | |
| b) | Confirm that coding sequence is authentic | | |
| | (1 week) | | |
| c) | Transfect HEK cells and confirm expression | | |
| | (1 week) | | |
| Step 2: | | 6 | weeks |
| a) | Select cell lines that are stably transfected | | |
| | (2 weeks) | | |
| b) | Adapt to suspension culture (up to 1 week) | | |
| c) | Adapt to Serum-free media (up to 1 week) | | |
| Step 3: | | 4-5 | weeks |
| a) | Scale up protein production to 1 L (2 weeks) | | |
| b) | Purify shTF to homogeneity, demonstrated by | | |
| | SDS gel (1 week) | | |

### Example 2

In this example the cloning of human soluble Tissue Factor is described in more details. Step 1: Cloning human soluble Tissue Factor (219aa) into an expression vector, transfection and expression of the protein

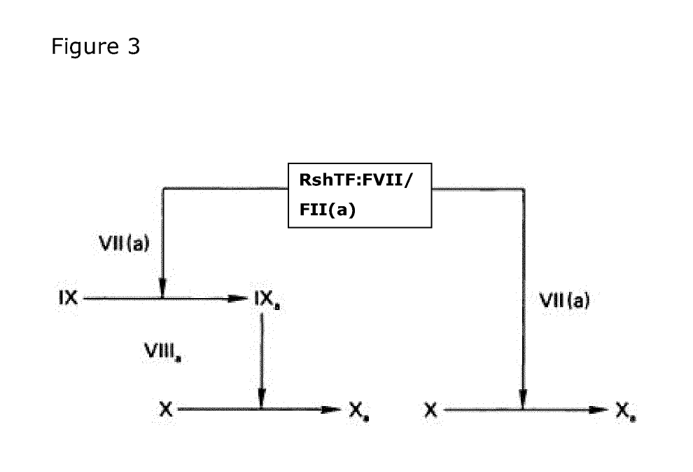

Gene source of human Tissue Factor (BC011029) was purchased from GE Lifesciences. Coding region of extracellular domain (soluble Tissue Factor; residues 1-219) was cloned into pST2 expression vector (Human Cell Co.) at Sill site to construct pST2-HF3 (FIGS. 1-3). After pST2-HF3 construction, soluble Tissue Factor coding region was sequenced and confirmed. pST2-HF3 was linearized with BglII and Pcil restriction enzymes for the plasmid to be integrated into the host chromosomal DNA after transfection.

Human embryonic kidney (HEK) cell handling was performed in a Laminar Hood (Nuaire NU-425-300). On day 1, 20 million suspension HEK293TS cells in CD293⁺ were plated on a 100 mm Petri dish in DMEM⁺ and grown to confluency in 24 hours. The next day the cells were passed to 5 dishes for transfection. After 24 hours growing, cells on one dish with 90% confluency were transfected with a mixture of linearized pST2-HF3 vector (10 μg), 500 μl DMEM medium without any supplement, and 30 μl polyethyleneimine, and grown in a water-jacketed humidified incubator (VWR 2310) with 5% CO₂ at 37° C.

### Example 3

Step 2: Cell Line Selection and Serum-Free and Suspension Adaptation

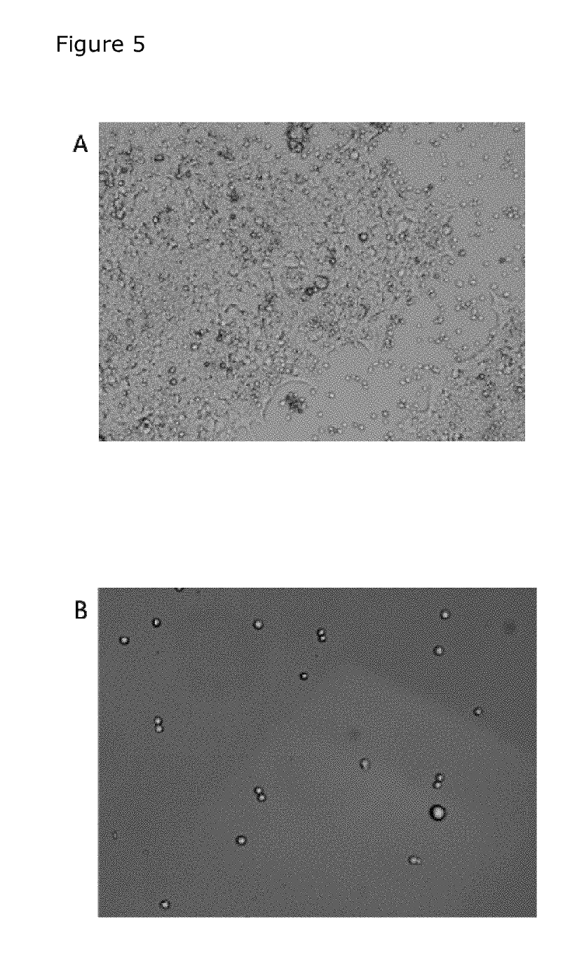

Two days culture after transfection, the cells were passed into three 100 mm Petri dishes with DMEM completed medium with 400 μg/mL zeocin (DMEM⁺ Z400) for selection. After one-week selection, survived cells from three dishes were pooled and transferred to a new plate and grown another week to confluency on the plate. The selected cells were then passed to 2 new plates with DMEM⁺ Z400. After 4 days when the cells reached confluency one plate was washed with 2 mL of PBS and treated with 1 mL of TrypLE for 5 minutes in the humidified incubator. After the incubation the cells were detached, collected by pipetting, and mixed with 1 mL of CD293 supplemented with 25 mM HEPES, 4 mM L-Gln, and 50 μg/mL zeocin (CD293⁺ Z50). The cell suspension was centrifuged at 200×g for 5 minutes and the supernatant was removed. The cell pellet was re-suspended in 10 mL CD293⁺ Z50 in a 125 mL plastic Erlenmeyer flask with a vent cap. The flask was moved onto a shaker (MAXQ2000, Thermo Scientific) rotating at 125 rpm in a dry CO₂ incubator with 5% CO₂ at 37° C. (Shel Lab 2428, Sheldon Manufacturing). Every 2-3 days the cell number was counted until the cells adapted to the serum-free chemically defined medium and culture. HEK293T cell morphologies in monolayer culture and in suspension culture are shown in FIG. 5.

### Example 4

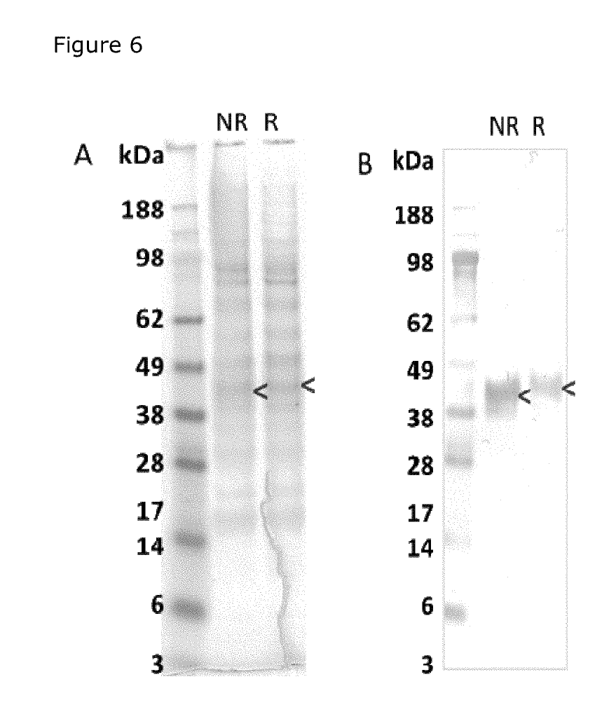

Over a 2 weeks period, the cells were adapted to the serum-free suspension culture and the soluble Tissue Factor band was identified on a SDS-PAGE gel (FIG. 6A) within 41-43 kDa due to glycosylation (derived from the human HEK293T cell line (theoretical molecular mass of the polypeptide without posttranslational modification is 24.8 kDa). The soluble Tissue Factor was also confirmed by Western blot as evidenced in (FIG. 6B) using an anti-human Tissue Factor monoclonal antibody (ab35807, Abcam).

### Example 5

Large scale production in 1 Litre batch was done in a pilot size Wave Bioreactor at a temperature of 37° C., starting as 100 mL culture with 0.5 million HEK 293T cells in suspension in serum-free medium, added during the week to 1000 ml as explained below.

Initial serum-free suspension adaptation of the selected cells showed 2-3 mg/L expression level of RshTF. While the protein purification protocol was developed, the cells were continuously cultured at 7 days batch culture mode in 100 mL culture with 0.5 million/mL seeding density in CD293⁺ Z50 for 4 more weeks.

After the extended culture, protein expression level was visibly improved, and the cells at the stage were cryobanked and set up for 1 L culture. From 2 days culture RshTF band was clearly visible on SDS-PAGE (FIG. 6A), and the viable cell number reached 3.8 million cells/mL with 87% viability after 7 days culture (FIG. 6B).

At day 9, the conditioned medium was harvested by centrifugation at 2,000×g for 10 minutes.

### Example 6

Currently known purification methods in the literature (see e.g. Broze, Jr. G J et al. (1985) J. Biol. Chem 260:10917-10920 or Guha A. et al. (1986) Proc. Natl. Acad. Sci. USA 83:299-302) are employing a Tissue Factor antibody or Tissue Factor VII affinity absorbent which are not applicable to large scale purification.

Therefore the inventors have developed a new protocol employing only conventional chromatography resins that are cheap and scalable with simple buffers.

Purification protocol was developed with AKTA FPLC, Unicorn software, pre-packed ion exchange and affinity columns (GE Lifesciences): On day 8 of 1 L culture, the cells were removed by centrifugation at 2,000×g for 10 minutes and the conditioned medium was harvested.

The conditioned medium was applied on 0.2 μm filter membrane to remove particles before loading onto the chromatography columns. As a virus inactivation process, Triton X-100 was added into the filtered medium at 1% final concentration. After 30 minutes incubation at room temperature, 2 volumes of WFI grade water was added to the medium and mixed to low the salt concentration to 50 mM ('5 mS/cm).

The low salt medium was loaded onto a HiTrap ANX FF column equilibrated with 10 mM Tris-HCl, pH 7.4. After the loading the column was washed with 2 column volumes of the equilibration buffer to get the baseline. Soluble Tissue Factor fraction was then eluted with 10 mM Tris-HCl, pH 7.4, 150 mM NaCl until the peak came down to the baseline. The column was stripped with 10 mM Tris-HCl, pH 7.4, 1 M NaCl.

### Example 7

The eluted soluble Tissue Factor fraction was mixed with 2 volumes of 10 mM Tris-HCl, pH 7.4 to low the salt concentration to 50 mM (˜5 mS/cm). Then the mixed fraction was loaded on HiTrap Heparin HP column equilibrated with 10 mM Tris-HCl, pH 7.4. The soluble Tissue Factor was in the flow through fraction. After the loading the column was washed with the equilibration buffer until the peak comes down to the baseline. The column was stripped with 10 mM Tris-HCl, pH 7.4, 1M NaCl (FIG. 9).

### Example 8

The soluble Tissue Factor fraction was then loaded onto HiTrap Q FF column equilibrated with 10 mM Tris-HCl, pH 7.4. After the loading the column was washed with 2 column volumes of the equilibration buffer to get the baseline. Soluble Tissue Factor fraction was then eluted with 10 mM Tris-HCl, pH 7.4, 300 mM NaCl until the peak came down to the baseline. The column was stripped with 10 mM Tris-HCl, pH 7.4, 1 M NaCl.

### Example 9

Purified soluble Tissue Factor fraction (210 mL) was processed buffer exchange to phosphate buffered saline (PBS) using Ultra 15 (MWCO 10 kDa, Millipore) according to the manufacturer's manual and 0.2 μm filter sterilized. Its concentration was measured by Agilent 8543 UV/vis spectrometer (FIG. 11A). On a SDS-PAGE gel soluble Tissue Factor was >95% pure (FIG. 11B). Total amount of purified and concentrated RshTF was 22.5 mg in 9 mL (2.5 mg/mL). Out of 9 mL, 1 mL (2.5 mg) was used for functional assay and 8 mL (20 mg) was placed at −80 ° C. for DGA.

### Example 10

Functional assays with fresh human blood.

Recombinant human Tissue Factor has been used as a key component in hemostasis in a number of prior art studies. The Tissue Factors currently used in the studies are produced in bacteria cells, and composed of extracellular domain and transmembrane domain.

However, the bacterial Tissue Factors are lacking of posttranslational modification (PTM) including glycosylation and disulphide bond due to the limitation of the expression system. Under such conditions exogenous phospholipid is also needed for proper function of the Tissue Factor.

The purpose of the functional assay was to test the functionality of RshTF produced with proper PTM from human cells in the human blood coagulation cascade without exogenous phospholipids supplied. A common outcome of the coagulation cascade between extrinsic and intrinsic pathways is producing active thrombin from prothrombin which is triggered by the exposed Tissue Factors at wound site. Calibrated Automated Thrombogram with Fluoroscan Ascent (Thermo Scientific) is a widely used instrument in research labs to monitor thrombin generation (said experiments were carried out at Washington University in St. Louis).

Each day a healthy donor donated 40 mL of blood with a nurse's help and calcium was chelated in the blood by sodium citrate added as an anticoagulant. The donated blood was transferred into 3.2% Citrate tubes and mixed by several gentle inversions.

The citrate mixed blood was centrifuged at 2,500×g for 15 minutes at 24° C. without break. After the centrifugation platelet-poor plasma (PPP) was gently collected using a polypropylene Pasteur's pipet. First day, we tested hematological function of rshTF in thrombin generation: 80 μl PPP was delivered into the designated wells in a 96 well plate and various concentrations of RshTF was added to the wells in 20 μl in duplicate.

Stock solution of RshTF is 2.5 mg/mL or 0.1 mM based on polypeptide molecular mass (24.8 kDa). Control well was added 20 μl of phosphate-buffered saline (PBS). To calculate thrombin generated, a known concentration of thrombin standard (Thrombin Calibrator, Stago) was set in triplicate: 80 μl PPP and 20 μl Thrombin Calibrator. After the PPP and RshTF were mixed in the plate, Fluka substrate solution (Stago), a thrombin substrate with fluorescent label, was automatically discharged 20 μl with calcium to the wells by the instrument and started thrombin generation reading.

At 10 nM RshTF concentration, thrombin generation started at 7 minutes and reached highest thrombin concentration (44 nM) at 19 minutes. At 1 nM and 0.1 nM, thrombin generation started at 15 and 33 minutes respectively. Interestingly, control with PBS showed thrombin generation at 1 hour albeit a significantly less amount (FIG. 13). From this study we concluded that RshTF alone can functionally initiate thrombin generation in platelet poor plasma without phospholipids provided, and that even PPP may have a little quantity of endogenous Tissue Factor. It is noteworthy that this may not reflect real physiological bleeding where all blood cells exist and calcium is not depleted.

### Example 11

On day two (continuing from example 10), instead of repeating the same experiment under a non-physiological condition, the inventors conducted a clot formation assay with whole blood. In each 100 μl of whole blood in a 1.5 mL Eppendorf tube, calcium was compensated at 10 mM. Clot formation was then triggered by adding a certain amount of RshTF, and mixing in the blood by shaking.

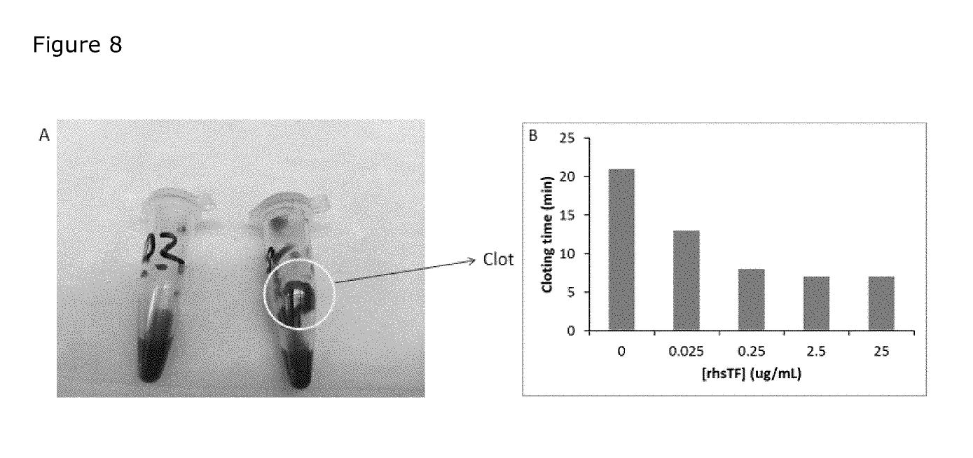

Then the inventors measured the time taken to form a clot. The result showed that RshTF triggered clot formation in the whole blood and did faster than PBS control: RshTF at 0.025 ug/mL formed a clot at 13 min whereas PBS control at 21 min (FIG. 8). Clot formation time was a dose-dependent response, and at 2.5 ug/ml clot formation time was shortened to 7 min (FIG. 8).

The conclusion the two experiments (examples 10-11) was that we RshTF alone triggered thrombin generation in PPP without exogenous phospholipids supplied and that RshTF in whole blood shortened the clot formation time in a dose-dependent manner. Additionally, the manner in which the RshTF cause the clot formation through its binding to Factor VII/VIIa to form RshTF:FVII/FVIIa. These encouraging findings justify homeostatic validation in animal models and in clinical trials.

Sequence listing

SEQ ID NO: 1 (nucleotide sequence encoding the soluble human Tissue Factor
(extracellular domain), HF3-663 nt):

| | |
|---|---|
| tcaggcacta caaatactgt ggcagcatat aatttaactt ggaaatcaac taatttcaag | 60 |
| acaattttgg agtgggaacc caaacccgtc aatcaagtct acactgttca aataagcact | 120 |
| aagtcaggag attggaaaag caaatgcttt tacacaacag acacagagtg tgacctcacc | 180 |
| gacgagattg tgaaggatgt gaagcagacg tacttggcac gggtcttctc ctacccggca | 240 |
| gggaatgtgg agagcaccgg ttctgctggg gagcctctgt atgagaactc cccagagttc | 300 |
| acaccttacc tggagacaaa cctcggacag ccaacaattc agagttttga acaggtggga | 360 |
| acaaaagtga atgtgaccgt agaagatgaa cggactttag tcagaaggaa caacactttc | 420 |
| ctaagcctcc gggatgtttt tggcaaggac ttaatttata cactttatta ttggaaatct | 480 |
| tcaagttcag gaaagaaaac agccaaaaca aacactaatg agtttttgat tgatgtggat | 540 |
| aaaggagaaa actactgttt cagtgttcaa gcagtgattc cctcccgaac agttaaccgg | 600 |
| aagagtacag acagcccggt agagtgtatg ggccaggaga aaggggaatt cagagaatga | 660 |
| taa | 663 |

```
SEQ ID NO: 2 (the amino acid sequence of the soluble human Tissue Factor
(extracellular domain (219 aa: Ser33 - Glu251)):
Ser Gly Thr Thr Asn Thr Val Ala Ala Tyr Asn Leu Thr Trp Lys Ser
1 5 10 15
Thr Asn Phe Lys Thr Ile Leu Glu Trp Glu Pro Lys Pro Val Asn Gln
                        20 25 30
Val Tyr Thr Val Gln Ile Ser Thr Lys Ser Gly Asp Trp Lys Ser Lys
                35 40 45
Cys Phe Tyr Thr Thr Asp Thr Glu Cys Asp Leu Thr Asp Glu Ile Val
        50 55 60
Lys Asp Val Lys Gln Thr Tyr Leu Ala Arg Val Phe Ser Tyr Pro Ala
65 70 75 80
Gly Asn Val Glu Ser Thr Gly Ser Ala Gly Glu Pro Leu Tyr Glu Asn
                                85 90 95
Ser Pro Glu Phe Thr Pro Tyr Leu Glu Thr Asn Leu Gly Gln Pro Thr
                        100 105 110
Ile Gln Ser Phe Glu Gln Val Gly Thr Lys Val Asn Val Thr Val Glu
                115 120 125
Asp Glu Arg Thr Leu Val Arg Arg Asn Asn Thr Phe Leu Ser Leu Arg
        130 135 140
Asp Val Phe Gly Lys Asp Leu Ile Tyr Thr Leu Tyr Tyr Trp Lys Ser
145 150 155 160
Ser Ser Ser Gly Lys Lys Thr Ala Lys Thr Asn Thr Asn Glu Phe Leu
                                165 170 175
Ile Asp Val Asp Lys Gly Glu Asn Tyr Cys Phe Ser Val Gln Ala Val
                        180 185 190
Ile Pro Ser Arg Thr Val Asn Arg Lys Ser Thr Asp Ser Pro Val Glu
                195 200 205
Cys Met Gly Gln Glu Lys Gly Glu Phe Arg Glu
        210 215
SEQ ID NO: 3 (nucleotide sequence (full sequence) of Human Tissue Factor (169-1056):
```

| | |
|---|---|
| atggagaccc ctgcctggcc ccgggtcccg cgccccgaga ccgccgtcgc tcggacgctc | 60 |
| ctgctcggct gggtcttcgc ccaggtggcc ggcgcttcag gcactacaaa tactgtggca | 120 |
| gcatataatt taacttggaa atcaactaat ttcaagacaa ttttggagtg ggaacccaaa | 180 |
| cccgtcaatc aagtctacac tgttcaaata agcactaagt caggagattg gaaaagcaaa | 240 |
| tgcttttaca caacagacac agagtgtgac ctcaccgacg agattgtgaa ggatgtgaag | 300 |
| cagacgtact tggcacgggt cttctcctac ccggcaggga atgtggagag caccggttct | 360 |
| gctggggagc ctctgtatga gaactcccca gagttcacac cttacctgga gacaaacctc | 420 |
| ggacagccaa caattcagag ttttgaacag gtgggaacaa aagtgaatgt gaccgtagaa | 480 |
| gatgaacgga ctttagtcag aaggaacaac actttcctaa gcctccggga tgtttttggc | 540 |
| aaggacttaa tttatacact ttattattgg aaatcttcaa gttcaggaaa gaaaacagcc | 600 |
| aaaacaaaca ctaatgagtt tttgattgat gtggataaag gagaaaacta ctgtttcagt | 660 |
| gttcaagcag tgattccctc ccgaacagtt aaccggaaga gtacagacag cccggtagag | 720 |
| tgtatgggcc aggagaaagg ggaattcaga gaaatattct acatcattgg agctgtggta | 780 |
| tttgtggtca tcatccttgt catcatcctg gctatatctc tacacaagtg tagaaaggca | 840 |
| ggagtggggc agagctggaa ggagaactcc ccactgaatg tttcataa | 888 |

```
SEQ ID NO: 4 (amino acid sequence of Human Tissue Factor full sequence (295 amino
acids)):
Met Glu Thr Pro Ala Trp Pro Arg Val Pro Arg Pro Glu Thr Ala Val
1 5 10 15
Ala Arg Thr Leu Leu Leu Gly Trp Val Phe Ala Gln Val Ala Gly Ala
                        20 25 30
Ser Gly Thr Thr Asn Thr Val Ala Ala Tyr Asn Leu Thr Trp Lys Ser
                35 40 45
Thr Asn Phe Lys Thr Ile Leu Glu Trp Glu Pro Lys Pro Val Asn Gln
        50 55 60
Val Tyr Thr Val Gln Ile Ser Thr Lys Ser Gly Asp Trp Lys Ser Lys
65 70 75 80
Cys Phe Tyr Thr Thr Asp Thr Glu Cys Asp Leu Thr Asp Glu Ile Val
                                85 90 95
Lys Asp Val Lys Gln Thr Tyr Leu Ala Arg Val Phe Ser Tyr Pro Ala
                        100 105 110
Gly Asn Val Glu Ser Thr Gly Ser Ala Gly Glu Pro Leu Tyr Glu Asn
                115 120 125
Ser Pro Glu Phe Thr Pro Tyr Leu Glu Thr Asn Leu Gly Gln Pro Thr
        130 135 140
Ile Gln Ser Phe Glu Gln Val Gly Thr Lys Val Asn Val Thr Val Glu
145 150 155 160
Asp Glu Arg Thr Leu Val Arg Arg Asn Asn Thr Phe Leu Ser Leu Arg
                                165 170 175
Asp Val Phe Gly Lys Asp Leu Ile Tyr Thr Leu Tyr Tyr Trp Lys Ser
                        180 185 190
Ser Ser Ser Gly Lys Lys Thr Ala Lys Thr Asn Thr Asn Glu Phe Leu
                195 200 205
Ile Asp Val Asp Lys Gly Glu Asn Tyr Cys Phe Ser Val Gln Ala Val
        210 215 220
Ile Pro Ser Arg Thr Val Asn Arg Lys Ser Thr Asp Ser Pro Val Glu
225 230 235 240
Cys Met Gly Gln Glu Lys Gly Glu Phe Arg Glu Ile Phe Tyr Ile Ile
                                245 250 255
Gly Ala Val Val Phe Val Val Ile Ile Leu Val Ile Ile Leu Ala Ile
                        260 265 270
Ser Leu His Lys Cys Arg Lys Ala Gly Val Gly Gln Ser Trp Lys Glu
                275 280 285
Asn Ser Pro Leu Asn Val Ser
        290 295
SEQ ID NO: 5 (nucleotide sequence of vector/plasmid pST2HF3 (sTF gene region (974 -
1636). The vector plasmid was linearized with BglII and PciI restriction enzymes)).
```

| | |
|---|---|
| gacggatcgg gagatctccc gatcccctat ggtcgactct cagtacaatc tgctctgatg | 60 |
| cgcatagtta agccagtatc tgctccctgc ttgtgtgttg gaggtcgctg agtagtgcgc | 120 |
| gagcaaaatt taagctacaa caaggcaagg cttgaccgac aattgcatga agaatctgct | 180 |
| tagggttagg cgttttgcgc tgcttcgcga tgtacgggcc agatatacgc gttgacattg | 240 |
| attattgact agttattaat agtaatcaat tacggggtca ttagttcata gcccatatat | 300 |
| ggagttccgc gttacataac ttacggtaaa tggcccgcct ggctgaccgc ccaacgaccc | 360 |
| ccgcccattg acgtcaataa tgacgtatgt tcccatagta acgccaatag ggactttcca | 420 |
| ttgacgtcaa tgggtggact atttacggta aactgcccac ttggcagtac atcaagtgta | 480 |
| tcatatgcca agtacgcccc ctattgacgt caatgacggt aaatggcccg cctggcatta | 540 |
| tgcccagtac atgaccttat gggactttcc tacttggcag tacatctacg tattagtcat | 600 |
| cgctattacc atggtgatgc ggttttggca gtacatcaat gggcgtggat agcggtttga | 660 |
| ctcacgggga tttccaagtc tccaccccat tgacgtcaat gggagtttgt tttggcacca | 720 |
| aaatcaacgg gactttccaa aatgtcgtaa caactccgcc ccattgacgc aaatgggcgg | 780 |
| taggcgtgta cggtgggagg tctatataag cagagctctc tggctaacta gagaacccac | 840 |
| tgcttactgg cttatcgaaa ttaatacgac tcactatagg gagacccaag ctggctagcc | 900 |
| accatggaga cagacacact cctgctatgg gtactgctgc tctgggttcc aggttccact | 960 |
| ggtgacgcgc ccgggccggc caggcgcgcg cgccgtacgt acgaagcttg gtaccgagct | 1020 |
| cggatccact ccagtgtggt ggaattctgc agatatccag cacagtggcg gccgctcgag | 1080 |
| gagggcccga acaaaaactc atctcagaag aggatctgaa tagcgccgtc gaccatcatc | 1140 |
| atcatcatca ttgagtttaa acccgctgat cagcctcgac tgtgccttct agttgccagc | 1200 |
| catctgttgt ttgcccctcc cccgtgcctt ccttgaccct ggaaggtgcc actcccactg | 1260 |
| tcctttccta ataaaatgag gaaattgcat cgcattgtct gagtaggtgt cattctattc | 1320 |
| tggggggtgg ggtggggcag gacagcaagg gggaggattg ggaagacaat agcaggcatg | 1380 |
| ctggggatgc ggtgggctct atggcttctg aggcggaaag aaccagctgg ggctctaggg | 1440 |
| ggtatcccca cgcgccctgt agcggcgcat taagcgcggc gggtgtggtg gttacgcgca | 1500 |
| gcgtgaccgc tacacttgcc agcgccctag cgcccgctcc tttcgctttc ttcccttcct | 1560 |
| ttctcgccac gttcgccggc tttccccgtc aagctctaaa tcggggcatc cctttagggt | 1620 |
| tccgatttag tgctttacgg cacctcgacc ccaaaaaact tgattagggt gatggttcac | 1680 |
| gtagtgggcc atcgccctga tagacggttt ttcgcccttt gacgttggag tccacgttct | 1740 |
| ttaatagtgg actcttgttc caaactggaa caacactcaa ccctatctcg gtctattctt | 1800 |
| ttgatttata agggattttg gggatttcgg cctattggtt aaaaaatgag ctgatttaac | 1860 |
| aaaaatttaa cgcgaattaa ttctgtggaa tgtgtgtcag ttagggtgtg gaaagtcccc | 1920 |
| aggctcccca gcaggcagaa gtatgcaaag catgcatctc aattagtcag caaccaggtg | 1980 |
| tggaaagtcc ccaggctccc cagcaggcag aagtatgcaa agcatgcatc tcaattagtc | 2040 |
| agcaaccata gtcccgcccc taactccgcc catcccgccc ctaactccgc ccagttccgc | 2100 |
| ccattctccg ccccatggct gactaatttt ttttatttat gcagaggccg aggccgcctc | 2160 |
| tgcctctgag ctattccaga agtagtgagg aggctttttt ggaggcctag gcttttgcaa | 2220 |
| aaagctcccg ggagcttgta tatccatttt cggatctgat cagcacgtgt tgacaattaa | 2280 |
| tcatcggcat agtatatcgg catagtataa tacgacaagg tgaggaacta aaccatggcc | 2340 |
| aagttgacca gtgccgttcc ggtgctcacc gcgcgcgacg tcgccggagc ggtcgagttc | 2400 |
| tggaccgacc ggctcgggtt ctcccgggac ttcgtggagg acgacttcgc cggtgtggtc | 2460 |
| cgggacgacg tgaccctgtt catcagcgcg gtccaggacc aggtggtgcc ggacaacacc | 2520 |
| ctggcctggg tgtgggtgcg cggcctggac gagctgtacg ccgagtggtc ggaggtcgtg | 2580 |
| tccacgaact tccgggacgc ctccgggccg gccatgaccg agatcggcga gcagccgtgg | 2640 |
| gggcgggagt tcgccctgcg cgacccggcc ggcaactgcg tgcacttcgt ggccgaggag | 2700 |
| caggactgac acgtgctacg agatttcgat tccaccgccg ccttctatga aaggttgggc | 2760 |
| ttcggaatcg ttttccggga cgccggctgg atgatcctcc agcgcgggga tctcatgctg | 2820 |
| gagttcttcg cccaccccaa cttgtttatt gcagcttata atggttacaa ataaagcaat | 2880 |
| agcatcacaa atttcacaaa taaagcattt ttttcactgc attctagttg tggtttgtcc | 2940 |
| aaactcatca atgtatctta tcatgtctgt ataccgtcga cctctagcta gagcttggcg | 3000 |
| taatcatggt catagctgtt tcctgtgtga aattgttatc cgctcacaat tccacacaac | 3060 |
| atacgagccg gaagcataaa gtgtaaagcc tggggtgcct aatgagtgag ctaactcaca | 3120 |
| ttaattgcgt tgcgctcact gcccgctttc cagtcgggaa acctgtcgtg ccagctgcat | 3180 |
| taatgaatcg gccaacgcgc ggggagaggc ggtttgcgta ttgggcgctc ttccgcttcc | 3240 |
| tcgctcactg actcgctgcg ctcggtcgtt cggctgcggc gagcggtatc agctcactca | 3300 |
| aaggcggtaa tacggttatc cacagaatca ggggataacg caggaaagaa catgtgagca | 3360 |
| aaaggccagc aaaaggccag gaaccgtaaa aaggccgcgt tgctggcgtt tttccatagg | 3420 |
| ctccgccccc ctgacgagca tcacaaaaat cgacgctcaa gtcagaggtg gcgaaacccg | 3480 |
| acaggactat aaagatacca ggcgtttccc cctggaagct ccctcgtgcg ctctcctgtt | 3540 |
| ccgaccctgc cgcttaccgg atacctgtcc gcctttctcc cttcgggaag cgtggcgctt | 3600 |
| tctcaatgct cacgctgtag gtatctcagt tcggtgtagg tcgttcgctc caagctgggc | 3660 |
| tgtgtgcacg aaccccccgt tcagcccgac cgctgcgcct tatccggtaa ctatcgtctt | 3720 |
| gagtccaacc cggtaagaca cgacttatcg ccactggcag cagccactgg taacaggatt | 3780 |
| agcagagcga ggtatgtagg cggtgctaca gagttcttga agtggtggcc taactacggc | 3840 |
| tacactagaa ggacagtatt tggtatctgc gctctgctga agccagttac cttcggaaaa | 3900 |
| agagttggta gctcttgatc cggcaaacaa accaccgctg gtagcggtgg tttttttgtt | 3960 |
| tgcaagcagc agattacgcg cagaaaaaaa ggatctcaag aagatccttt gatcttttct | 4020 |
| acggggtctg acgctcagtg gaacgaaaac tcacgttaag ggattttggt catgagatta | 4080 |
| tcaaaaagga tcttcaccta gatcctttta aattaaaaat gaagttttaa atcaatctaa | 4140 |
| agtatatatg agtaaacttg gtctgacagt taccaatgct taatcagtga ggcacctatc | 4200 |
| tcagcgatct gtctatttcg ttcatccata gttgcctgac tccccgtcgt gtagataact | 4260 |
| acgatacggg agggcttacc atctggcccc agtgctgcaa tgataccgcg agacccacgc | 4320 |
| tcaccggctc cagatttatc agcaataaac cagccagccg gaagggccga gcgcagaagt | 4380 |
| ggtcctgcaa ctttatccgc ctccatccag tctattaatt gttgccggga agctagagta | 4440 |
| agtagttcgc cagttaatag tttgcgcaac gttgttgcca ttgctacagg catcgtggtg | 4500 |
| tcacgctcgt cgtttggtat ggcttcattc agctccggtt cccaacgatc aaggcgagtt | 4560 |
| acatgatccc ccatgttgtg caaaaaagcg gttagctcct tcggtcctcc gatcgttgtc | 4620 |
| agaagtaagt tggccgcagt gttatcactc atggttatgg cagcactgca taattctctt | 4680 |
| actgtcatgc catccgtaag atgcttttct gtgactggtg agtactcaac caagtcattc | 4740 |
| tgagaatagt gtatgcggcg accgagttgc tcttgcccgg cgtcaatacg ggataatacc | 4800 |
| gcgccacata gcagaacttt aaaagtgctc atcattggaa aacgttcttc ggggcgaaaa | 4860 |
| ctctcaagga tcttaccgct gttgagatcc agttcgatgt aacccactcg tgcacccaac | 4920 |
| tgatcttcag catcttttac tttcaccagc gtttctgggt gagcaaaaac aggaaggcaa | 4980 |
| aatgccgcaa aaaagggaat aagggcgaca cggaaatgtt gaatactcat actcttcctt | 5040 |
| tttcaatatt attgaagcat ttatcagggt tattgtctca tgagcggata catatttgaa | 5100 |
| tgtatttaga aaaataaaca aataggggtt ccgcgcacat ttccccgaaa agtgccacct | 5160 |
| gacgtc | 5166 |

1. A recombinant soluble human Tissue Factor (RshTF) comprising the SEQ ID NO: 2, wherein said recombinant soluble human Tissue Factor is produced in a human HEK293 cell.

2. The recombinant soluble human Tissue Factor according to claim 1, wherein the human HEK293 cell is selected from the group consisting human HEK293T cells, human HEK293TS cells and human HEK293E cells.

3. The recombinant soluble human Tissue Factor according to claim 1, wherein the recombinant soluble human Tissue Factor is essentially serum-free.

4. The recombinant soluble human Tissue Factor according to claim 1, wherein the recombinant soluble human Tissue Factor is glycosylated with one or more non-reducing disaccharides, preferably trehalose, and/or with one or more disaccharides consisting of sugar-alcohol combinations, preferably mannitol.

5. The recombinant soluble human Tissue Factor according to claim 1, wherein at least 90% of the recombinant soluble human Tissue Factor comprises a glycolysation pattern that results in an immunogenicity response substantially identical to that of the corresponding natural human Tissue Factor.

6. The recombinant soluble human Tissue Factor according to claim 1, wherein said recombinant soluble human Tissue Factor is part of a glue, a powder, a spray, a gel, a granulate, a gelatin, a paste, a fluid, a suture, a scaffold, a paste and/or a cream substance to which said Factor can be bound or mixed.

7. The recombinant soluble human Tissue Factor according to claim 6, wherein the powder is MPEG PLGA.

8. The recombinant soluble human Tissue Factor according to claim 6, wherein the scaffold is a haemostatic scaffold in the form of a gel, a foam, polyurethane foam, a gelatin-like substance, a collagen, a collagen-derived paste and/or a gelatin-derived paste.

9. The recombinant soluble human Tissue Factor according to claim 8, wherein the concentration of recombinant soluble human Tissue Factor on the scaffold range from 0.1 μg-1000 ug per cm² scaffold.

10. The recombinant soluble human Tissue Factor according to claim 1, wherein the recombinant soluble human Tissue Factor is in a solution in a concentration in the range from 0.1 μg/ml-1000 μg/ml.

11-27. (canceled)

28. Method of treating and/or preventing bleedings in humans comprising topical administration on the bleeding site in a human with a recombinant soluble human Tissue Factor according to claim 1.

29. A method of producing recombinant soluble human Tissue Factor (RshTF), said method comprising culturing in vitro a human host cell transfected with at least one exogenous nucleic acid sequence encoding said recombinant soluble human Tissue Factor, wherein the human host cell is a human HEK293 cell.

30. The method according to claim 29, wherein the human HEK293 cell is selected from the group consisting of human HEK293T cells, human HEK293E cells and human HEK293TS cells.

31. The method according to claim 29, wherein the in vitro culturing is carried out under essentially serum-free conditions.

32. The method according to claim 29, wherein the transfected human HEK cell is cultured to a cell density between 3-9 million cells/mL culture medium.

33. The method according to claim 29, wherein the recombinant soluble human Tissue Factor is essentially serum-free.

34. The method according to claim 29, wherein the exogenous nucleic acid sequence comprises SEQ ID NO: 1.

35. (canceled)

36. The method according to claim 29, wherein the human HEK293 cell is human HEK293TS cells.

37. The recombinant soluble human Tissue Factor according to claim 1, wherein the human HEK293 cell is HEK293TS cells.


SEQUENCE LISTING (5 sequences)

SEQ ID NO: 1 - DNA, 663 bp, Homo sapiens
tcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaatgataa

SEQ ID NO: 2 - protein, 219 aa, Homo sapiens
  MISC_FEATURE (1)..(219)
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 3 - DNA, 888 bp, Homo sapiens
  misc_feature (1)..(888)
atggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataa

SEQ ID NO: 4 - protein, 295 aa, Homo sapiens
  MISC_FEATURE (1)..(295)
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 5 - DNA, 5166 bp, Artificial sequence
  misc_feature (1)..(5166): Synthesized, linearized by Bg III and Pcil restriction enzymes
gacggatcgggagatctcccgatcccctatggtcgactctcagtacaatctgctctgatgcgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcgcccgggccggccaggcgcgcgcgccgtacgtacgaagcttggtaccgagctcggatccactccagtgtggtggaattctgcagatatccagcacagtggcggccgctcgaggagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc